SEQUENCE LISTING (26 sequences)

SEQ ID NO: 1 - protein, 9 aa, Artificial Sequence
  CRE motif
LAGLIDADG

SEQ ID NO: 2 - DNA, 22 bp, Artificial Sequence
  CRE Recognition sequence
caaaacgtcgtgagacagtttg

SEQ ID NO: 3 - DNA, 132 bp, Artificial sequence
  PEST sequence
  CDS (1)..(132)
aagcttagccatggcttcccgccggaggtggaggagcaggatgatggcacgctgcccatgtcttgtgcccaggagagcgggatggaccgtcaccctgcagcctgtgcttctgctaggatcaatgtgtagtaa

SEQ ID NO: 4 - protein, 42 aa, Artificial sequence
  Synthetic Construct
KLSHGFPPEVEEQDDGTLPMSCAQESGMDRHPAACASARINV

SEQ ID NO: 5 - DNA, 22 bp, Artificial sequence
  PCSK9 target
tggacctctttgccccagggga

SEQ ID NO: 6 - DNA, 22 bp, Artificial sequence
  Mutant Target sequence for PCSK9 meganuclease
ttgccctttttattcccaggga

SEQ ID NO: 7 - DNA, 22 bp, Artificial Sequence
  TTR Target
gctggactggtatttgtgtctg

SEQ ID NO: 8 - DNA, 22 bp, Artificial Sequence
  TTR Mutant Target
tcgggacttttgtttgcctctt

SEQ ID NO: 9 - DNA, 2625 bp, Artificial sequence
  PCSK7-8L.197
  CDS (1089)..(2183): M2PCSK9 coding sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - protein, 364 aa, Artificial sequence
  Synthetic Construct
MAPKKKRKVHMNTKYNKEFLLYLAGFVDGDGSIFARIKPSQRSKFKHKLHLVFAVYQKTQRRWFLDKLVDEIGVGYVLDSGSVSFYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIKPVQRAKFKHELVLGFDVTQKTQRRWFLDKLVDEIGVGYVYDKGSVSAYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 11 - DNA, 2642 bp, Artificial sequence
  TBG.Target.PI.M2PCSK9 Nuclease.bGH
  misc_feature (1)..(168): ITR
  misc_feature (211)..(907): Promoter/enhancer
  misc_feature (914)..(395): Target
  misc_feature (961)..(1093): Intron
  misc_feature (1110)..(2204): M2PCKS9 CDS
  misc_feature (2211)..(2425): bovine growth hormone (bGH) polyA
  misc_feature (2475)..(2642): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagtggacctctttgccccaggggaaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 2764 bp, Artificial sequence
  TBG.Target.PI.PCS9 Nuclease-PEST
  misc_feature (1)..(168): ITR
  misc_feature (211)..(907): promoter/enhancer
  misc_feature (914)..(935): target
  misc_feature (961)..(1093): intron
  misc_feature (1109)..(2200): M2PCSK9 CDS
  misc_feature (2201)..(2332): PEST
  misc_feature (2333)..(2547): bovine growth hormone (bGH) polyA
  misc_feature (2597)..(2764): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagtggacctctttgccccaggggaaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccacggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtcccccaagcttagccatggcttcccgccggaggtggaggagcaggatgatggcacgctgcccatgtcttgtgcccaggagagcgggatggaccgtcaccctgcagcctgtgcttctgctaggatcaatgtgtagtaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 2792 bp, Artificial sequence
  TBG.Target.PI.PCS9 Nuclease-PEST.Target.bGH
  repeat_region (1)..(168): ITR
  promoter (211)..(907): TBG promoter
  enhancer (211)..(310): alpha/mic/bik enhancer
  enhancer (317)..(416): enhancer 2
  misc_feature (914)..(935): Target sequence
  Intron (961)..(1093)
  misc_feature (1110)..(2201): PCSK7-8L.197 coding sequence
  misc_feature (2202)..(2330): PEST
  misc_feature (2334)..(2355): target
  polyA_signal (2361)..(2575): bovine growth hormone (bGH) polyA
  repeat_region (2625)..(2792): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagtggacctctttgccccaggggaaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtcccccaagcttagccatggcttcccgccggaggtggaggagcaggatgatggcacgctgcccatgtcttgtgcccaggagagcgggatggaccgtcaccctgcagcctgtgcttctgctaggatcaatgtgtagtaatggacctctttgccccaggggagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 2765 bp, Artificial Sequence
  TBG.MutTarget.PI.Nuclease-PEST.bGH
  misc_feature (1)..(168): ITR
  misc_feature (211)..(907): Promoter/enhancer
  misc_feature (914)..(935): mutant target
  misc_feature (961)..(1093): intron
  misc_feature (1110)..(2201): M2PSCK9 CDS
  misc_feature (2202)..(2333): PEST CDS
  misc_feature (2334)..(2548): bGH Poly A
  misc_feature (2598)..(2765): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagttgccctttttattcccagggaaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtcccccaagcttagccatggcttcccgccggaggtggaggagcaggatgatggcacgctgcccatgtcttgtgcccaggagagcgggatggaccgtcaccctgcagcctgtgcttctgctaggatcaatgtgtagtaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 2744 bp, Artificial sequence
  TBG.PI.PCSK9 Nuclease-PEST
  misc_feature (1)..(168): ITR
  misc_feature (211)..(907): Promoter/enhancer
  misc_feature (939)..(1071): intron
  misc_feature (1089)..(2180): M2PCSK9 CDS
  misc_feature (2181)..(2312): PEST CDS
  misc_feature (2313)..(2527): bGH Poly A
  misc_feature (2577)..(2744): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtcccccaagcttagccatggcttcccgccggaggtggaggagcaggatgatggcacgctgcccatgtcttgtgcccaggagagcgggatggaccgtcaccctgcagcctgtgcttctgctaggatcaatgtgtagtaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 2646 bp, Artificial sequence
  Vector genome TBG.MutTarget.PI.PCS9 Nuclease.bGH
  misc_feature (1)..(168): ITR
  misc_feature (211)..(907): promoter/enhancer
  misc_feature (914)..(935): mutant target
  misc_feature (961)..(1093): intron
  misc_feature (1110)..(2204): M2PCSK9 CDS
  misc_feature (2215)..(2429): bGH Poly A
  misc_feature (2479)..(2646): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagttgccctttttattcccagggaaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 1224 bp, Artificial sequence
  PCSK9 Meganuclease - PEST fusion
  CDS (1)..(1224)
atggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtcccccaagcttagccatggcttcccgccggaggtggaggagcaggatgatggcacgctgcccatgtcttgtgcccaggagagcgggatggaccgtcaccctgcagcctgtgcttctgctaggatcaatgtgtagtaa

SEQ ID NO: 18 - protein, 406 aa, Artificial sequence
  Synthetic Construct
MAPKKKRKVHMNTKYNKEFLLYLAGFVDGDGSIFARIKPSQRSKFKHKLHLVFAVYQKTQRRWFLDKLVDEIGVGYVLDSGSVSFYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIKPVQRAKFKHELVLGFDVTQKTQRRWFLDKLVDEIGVGYVYDKGSVSAYRLSQIKPLHNFLTQLQPFLKLKQKQANLVLKIIEQLPSAKESPDKFLEVCTWVDQIAALNDSKTRKTTSETVRAVLDSLSEKKKSSPKLSHGFPPEVEEQDDGTLPMSCAQESGMDRHPAACASARINV

SEQ ID NO: 19 - DNA, 23 bp, Artificial Sequence
  Primer
tgctttgttcctcttggccttgg

SEQ ID NO: 20 - DNA, 22 bp, Artificial Sequence
  Primer
tcctggcaaagatggaaccgtc

SEQ ID NO: 21 - DNA, 26 bp, Artificial Sequence
  Primer
gactccaagacccgcaagaccacttc

SEQ ID NO: 22 - DNA, 30 bp, Artificial Sequence
  Primer
ttattaggaaaggacagtgggagtggcacc

SEQ ID NO: 23 - DNA, 23 bp, Artificial Sequence
  Primer
tgccacctacctcctcacctttc

SEQ ID NO: 24 - DNA, 20 bp, Artificial Sequence
  Primer
ggctgttagcatcacggtgg

SEQ ID NO: 25 - DNA, 27 bp, Artificial Sequence
  Primer
cctcagctcccgaggtcatcacagttg

SEQ ID NO: 26 - DNA, 30 bp, Artificial Sequence
  Primer
tgacatctttggcagagaagtggatcagtc

(19) United States
(12) Patent Application Publication
(10) Pub. No.: US 2022/0298500 A1
(43) Pub. Date: Sep. 22, 2022

(54) COMPOSITIONS FOR REGULATING AND SELF-INACTIVATING ENZYME EXPRESSION AND METHODS FOR MODULATING OFF-TARGET ACTIVITY OF ENZYMES

(71) Applicant: The Trustees of the University of Pennsylvania, Philadelphia, PA (US)

(72) Inventors: James M. Wilson, Philadelphia, PA (US); Camilo Breton, St. Davids, PA (US)

(21) Appl. No.: 17/603,993

(22) PCT Filed: Apr. 15, 2020
(86) PCT No.: PCT/US2020/028344

§ 371 Date: Oct. 15, 2021

Related U.S. Application Data
(60) Provisional application No. 62/834,064, filed on Apr. 15, 2019.

(51) Int. Cl. C12N 15/10; C12N 15/86; C12N 9/22

(52) U.S. Cl. CPC C12N 15/102; C12N 15/86; C12N 9/22; C07K 2319/09; C07K 2319/80; C07K 2319/95; C12N 2750/14143

(57) ABSTRACT

A gene editing nuclease expression cassette is provided which comprises a nucleic acid sequence comprising an nuclease coding sequence which is operably linked to regulatory sequences which direct expression of the nuclease following delivery to a host cell having a sequence to which the nuclease is targeted and at least one nuclease modulating sequence which is selected from the target sequence for the nuclease or a mutated target sequence which is recognized by the nuclease following its expression. A vector is provided comprising the gene editing nuclease expression cassette. Also provided are compositions containing same and methods of use.

### BACKGROUND OF THE INVENTION

The use of engineered nucleases has been described for editing dysfunctional genes. AAV-mediated delivery of such nucleases has also been described. However, while AAV-mediated delivery of nucleases avoids the need for repeated readministration, the resulting nuclease it is continuously expressed in the target tissue following vector transduction, which may induce immune responses and cellular toxicity.

Further, both in vitro and in vivo studies have shown that nucleases, regardless of delivery vehicle, generate indels (insertions and deletions) in other regions of the genome, suggesting off-target activity.

What are needed are improved compositions and methods for gene editing.

### SUMMARY OF THE INVENTION

A delivery system for enzymes is provided which provides a reduction of off-target activity, thus improving safety of the delivered enzyme. The system is particularly well suited for use in gene editing therapies and/or in enzymes delivered by systems in which the gene persists in the cells, such as AAV-mediated delivery.

In one aspect, a self-modulating gene editing nuclease expression cassette is provided. The expression cassette comprises (a) a nucleic acid sequence comprising a nuclease coding sequence which is operably linked to regulatory sequences which direct expression of the nuclease following delivery to a host cell having a sequence to which the nuclease is targeted; and (b) at least one nuclease modulating sequence which is selected from the target sequence for the nuclease or a mutated target sequence which is recognized by the nuclease following its expression. Optionally, the coding sequence encodes a fusion protein comprising at least one peptide degradation signal in frame with the nuclease coding sequence. In certain embodiments, the protein degradation signal is 10 amino acids to 50 amino acids in length. In certain embodiments, the protein degradation sequence is a PEST sequence of about 42 amino acids in length. In certain embodiments, the expression cassette comprises more than one protein degradation signal. In certain embodiments, the expression cassette comprises a mutated target sequence, which may be located upstream, downstream or within the nuclease coding sequence. In embodiments where the expression cassette has two or more nuclease modulating sequences, they may be independently located and may the same or different sequences. In certain embodiments, the self-modulating nuclease expression cassette may have more than one protein degradation signal.

In certain embodiments, a self-inactivating meganuclease expression cassette is provided which comprises a nucleic acid sequence comprising sequence encoding a meganuclease fused to at least one protein degradation sequence and regulatory sequences which direct expression of the meganuclease and protein degradation signal as fusion protein in a host cell. In certain embodiments, the protein degradation signal is 10 amino acids to 50 amino acids in length. In certain embodiments, the protein degradation sequence is a PEST sequence of about 42 amino acids in length. In certain embodiments, the expression cassette comprises more than one protein degradation signal.

In certain embodiments, a recombinant AAV useful for gene editing comprising an AAV capsid and a vector genome packaged in the AAV capsid, said vector genome comprising an expression cassette as described in the preceding paragraphs and AAV inverted terminal repeat (ITR) sequences required for packaging the expression cassette into the capsid.

A pharmaceutical composition comprising an expression cassette as described herein is provided and one or more of a carrier, diluent, and/or excipient. In certain embodiments, the expression cassette is in a non-vector based delivery system, e.g., a lipid nanoparticle (LNP). In certain embodiments, the expression cassette is engineered into a non-viral vector (e.g., a plasmid or other genetic element), which recombinant vector is admixed with a carrier, diluent and/or excipient to form the pharmaceutical composition. In certain embodiments, the expression cassette is engineered into a viral vector, which recombinant vector is admixed with a carrier, diluent and/or excipient to form the pharmaceutical composition. In certain embodiments, the expression cassette is engineered into a vector genome and packaged into an AAV capsid to form a recombinant adeno-associated virus (rAAV), which rAAV is admixed with a carrier, diluent and/or excipient to form the pharmaceutical composition.

In certain embodiments, a nuclease expression cassette, non-viral vector (e.g., DNA or RNA), or viral vector (e.g.,rAAV or lentivirus), as described herein is administrable for gene editing in a patient. In certain embodiments, the method is useful for non-embryonic gene editing. In certain embodiments, the patient is an infant (e.g., birth to about 9 months). In certain embodiments, the patient is older than an infant, e.g, 12 months or older.

In certain embodiments, a pharmaceutical composition as described herein is administrable for gene editing in a patient. In certain embodiments, the method is useful for non-embryonic gene editing. In certain embodiments, the patient is an infant (e.g., birth to about 9 months). In certain embodiments, the patient is older than an infant, e.g, 12 months or older.

Other aspects and advantages of the invention will be apparent from the following detailed description of the invention.

### BRIEF DESCRIPTION OF THE DRAWINGS

FIG. 1 is a schematic representation of AAV “suicide” vectors. AAV vectors were constructed by inserting the meganuclease (M2PCSK9) with a combination of the target sequence (dark bar with *), mutant target sequence containing 8 mismatches (dark bar with *mut), and a PEST sequence (black bar with lines).

FIG. 2A shows a timeline of in vivo experiments. Mouse study. Human PCSK9 (hPCSK9)-expressing AAV was intravenously (IV) injected in RAG KO mice. Two weeks later, a single dose of the AAV suicide vectors or corresponding control (AAV8.M2PCSK9) were IV injected into treated mice. Non-human primate study. AAV.MutTarget.M2PCSK9-PEST or AAV.M2PCSK9 were administered into rhesus macaques. Liver biopsies were collected at day 18 or day 128 post-treatment.

FIG. 2B shows indels in the target region in purified AAV vectors.

FIG. 3A (left) and FIG. 3B (right) shows meganuclease-induced indels in hPCSK9 gene and in AAV suicide vectors. Mice were treated with the indicated AAV vector and euthanized at the indicated time post-AAV9.hPCSK9 administration. Indel % for each target is shown as percentage of total reads.

FIG. 4A (left) and FIG. 4B (right) show the in vivo off-target activity of the meganuclease suicide system. Number of unique AAV integration sites in the host genome were identified and quantified in liver DNA samples obtained at 4 and 9 weeks post AAV9.hPCSK9 administration (left). From the obtained list, nine of the most common off-targets locations were selected and the indel % in these loci was calculated by NGS and plotted as relative to meganuclease-only control (right).

FIGS. 5A-5I show results when AAV suicide vectors were administered to rhesus macaques. PCSK9 (FIG. 5A) and LDL protein levels (FIG. 5B) in serum samples at different times post-vector administration. Indel % in the target sequence within the PCSK9 gene as detected by Amplicon-Seq (FIG. 5C) or AMP-Seq methods (FIG. 5D) and number of unique off-targets sites (FIG. 5E) at day 18 post-vector administration (d18). AAV genome copy numbers and meganuclease mRNA levels in NHP livers at d18 (FIG. 5F). In situ hybridization in liver sections obtained from a liver biopsy at d18 using a probe specific for the meganuclease DNA/mRNA (FIG. 5G). The editing in the PCSK9 target region induced a decrease in PCSK9 as previously observed (Nat Biotechnol. 2018 September;36(8):717-725), which also lead to a reduction in the LDL levels at different times post vector injection (FIG. 5H and 5I, respectively).

FIG. 6 is a schematic representation of AAV.TTR “suicide” vectors. AAV vectors were constructed by inserting the meganuclease with a combination of the target sequence, mutant target sequence containing 8 mismatches, and a PEST sequence.

FIGS. 7A-7C provide a sequence analysis of AAV ITRs integrated into genomic DNA. FIG. 7A is a meta-analysis of on-target AMP-Seq data for all AAV8-M1PCSK9 and AAV8-M2PCSK9 treated liver samples (SRR6343442). Our goal was to identify the most frequent ITR integration start site within the vector ITR. FIG. 7B shows the secondary structure of the AAV2 5′ ITR (NC_001401.2). The most frequently integrated start site position is shown. ITR-Seq primer GSP_ITR3.AAV2 binding site is highlighted in red. A-A′, B-B′ and C-C′, palindromic arms; RBE, rep-binding element; TRS, terminal resolution site. FIG. 7C is a schematic diagram of the ITR-Seq protocol used for genome-wide identification of ITR integration sites.

FIGS. 8A-8C show analysis of on- and off-target activity of AAV8-M1PCSK9 and AAV8-M2PCSK9 in vivo. FIG. 8A is ITR-Seq identified integration sites for AAV8-M1PCSK9 and AAV8-M2PCSK9 treated liver samples collected at 17 and 128 days following vector administration. FIG. 8B is a functional annotation of ITR identified ITR integration sites, showing the number of sites within exons, introns, intergenic, transcription start sites (TSS), and transcription termination sites (TTS). FIG. 8C shows distribution of ITR integration sites at day 17/18 for 2 animals treated with either M1PCSK9 or M2PCSK9 (bars) or for computationally generated random DNA sequences (dotted line) according to the number of nucleotides that match the intended target sequence, represented as a percentage of the total number of identified sites

FIGS. 9A-9D show a comparison of GUIDE-Seq and ITR-Seq identified off-targets. Sample set intersections of identified target sites obtained from in vivo ITR-Seq (left sections) from AAV8-M1PCSK9 at 3×10¹³ GC/kg (FIG. 9A), at 6×10¹² GC/kg (FIG. 9B) or AAV8-M2PCSK9 at 6×10¹² GC/kg (FIG. 9C and FIG. 9D) at day 17 post-AAV administration and in vitro GUIDE-Seq for M1PCSK9 or M2PCSK9 (right sections). Off-targets identified by ITR-Seq but not GUIDE-Seq (left sections) are indicated as a percentage of the total number of off-targets identified by in vivo ITR-Seq. Off-targets identified by both ITR-Seq and GUIDE-Seq are shown as white sections of the Venn diagrams.

FIGS. 10A-10B show embodiments in which a target sequence or mutant target sequence is inserted in the enzyme coding sequence. FIG. 10A is an amino acid alignment showing a 10-amino acid nuclear localization signal (NLS) followed by the protein expressed from a target sequence (amino acids 10-20, followed by the active portion of the nuclease. The first amino acid sequence M2PCSK9 shows a fragment of a reference meganuclease with its NLS, a space noting where the other constructs will have insertions, and the sequence of the nuclease beginning at position 18. M2PCSK9[inverted target #1] shows the protein encoded when a target sequence is inserted on the anti-sense strand between the NLS and the enzyme. M2PCSK9[target #1] shows the protein encoded when a target sequence inserted on the sense strand between the NLS and the enzyme. M2PCSK9 [target #2] shows the protein encoded when a different target sequence is inserted on the sense strand between the NLS and the enzyme. M2PCSK9 [inverted target #2] shows the protein encoded when the (mut)target sequence is inserted on the anti-sense strand between the NLS and the enzyme. [target]M2PCSK9 shows the protein encoded when the 22 bp target sequence replaces the coding sequences after the NLS, resulting in substitution of the six amino acids of the enzyme. [invertedtarget]M2PCSK9 shows the protein encoded when the 22 bp target sequence replaces the coding sequences after the NLS on the opposite strand, resulting in replacement of the seven amino acids of the enzyme. FIG. 10B shows the design of a PCSK9 meganuclease fusion protein, one having a single protein degradation signal (degron) which is ubiquitin-independent (AR-6), and a PCSK9 meganuclease fusion protein having two protein degradation signals, AR-6 and PEST.

### DETAILED DESCRIPTION OF THE INVENTION

The compositions and methods provided herein are designed to minimize off-target activity of a persistently expressed enzyme (e.g., following delivery of an expression cassette) and/or modulating the activity of the expressed enzyme. Use of these compositions and methods with non-secreting enzymes which may accumulate in a cell and/or enzymes which accumulate at higher than desired levels prior to secretion is particularly desirable. The compositions and methods of the invention are well suited for use with gene editing enzymes, particularly meganucleases. However, other applications will be apparent to one of skill in the art.

As used herein, a suitable enzyme may be selected from meganucleases, zinc finger nucleases, transcription activator-like (TAL) effector nucleases (TALENs), a clustered, regularly interspaced short palindromic repeat (CRISPR)/endonuclease (Cas9, Cpfl, etc). Examples of suitable meganucleases are described, e.g., in U.S. Pat. Nos. 8,445,251; 9,340,777; 9,434,931; 9,683,257, and WO 2018/195449. Other suitable enzymes include nuclease-inactive S. pyogenes CRISPR/Cas9 that can bind RNA in a nucleic-acid-programmed manner (Nelles et al, Programmable RNA Tracking in Live Cells with CRISPR/Cas9, Cell, 165(2):P488-96 (April 2016)), and base editors (e.g., Levy et al. Cytosine and adenine base editing of the brain, liver, retina, heart and skeletal muscle of mice via adeno-associated viruses, Nature Biomedical Engineering, 4, 97-110 (Jan 2020)). In certain embodiments, the nuclease is not a zinc finger nuclease. In certain embodiments, the nuclease is not a CRISPR-associated nuclease. In certain embodiments, the nuclease is not a TALEN.

In certain embodiments, the nuclease is a member of the LAGLIDADG (SEQ ID NO: 1) family of homing endonucleases. In certain embodiments, the nuclease is a member of the I-CreI family of homing endonucleases which recognizes and cuts a 22 base pair recognition sequence SEQ ID NO: 2—CAAAACGTCGTGAGACAGTTTG. See, e.g., WO 2009/059195. Methods for rationally-designing mono-LAGLIDADG homing endonucleases were described which are capable of comprehensively redesigning ICrel and other homing endonucleases to target widely-divergent DNA sites, including sites in mammalian, yeast, plant, bacterial, and viral genomes (WO 2007/047859).

Suitably, the coding sequence for a nuclease a described herein is engineered into an expression cassette which further contains either the self-inactivating components, the self-modulating components, or both, operably linked to regulatory elements which direct expression of the enzyme or enzyme — protein degradation signal (degron) in the cell containing the target site for the enzyme.

### Self-Inactivating Nuclease Fusion Protein

In one embodiment, a self-inactivating nuclease expression cassette contains the coding sequence for nuclease fused in frame with a protein degradation signal such that a fusion protein is produced which comprises a nuclease and at least one protein degradation signal. In one embodiment, a self-inactivating nuclease expression cassette contains the coding sequence for the meganuclease fused in frame with a protein degradation signal such that a fusion protein is produced which comprises a meganuclease and at least one protein degradation signal. For convenience, the hyphenated phrase term “enzyme—protein degradation signal” is used to refer to such fusion proteins. This is not intended to limit the fusion protein to the protein degradation signal being located at the carboxy terminus, or to only one such protein degradation signal being present in the fusion protein. Similarly, it will be understood that a particular type of enzyme may be specified, e.g., “nuclease”, “meganuclease”, “endonuclease” and the like, in the above phrase and is subject to the same interpretation with respect to the location and number of the protein degradation signal not being limited, unless it is specified.

As described herein, a protein degradation signal is a portion of a protein which mediates the degradation of the enzyme, which may also be termed a degron. Without wishing to be bound by theory, it is believed that a fusion protein containing the enzyme (e.g., nuclease) and at least one protein degradation signal reduces off-target activity without compromising on-target efficacy by promoting removal of the enzyme from the cell when the presence of its natural substrate (target sequence) has been reduced by the enzymatic activity of the fusion protein. The protein degradation signal reduces the half-life of the protein and therefore also reduces the levels of accumulated protein. This decrease in protein levels, is believed to help reduce the nuclease off-target activity. The peptide degradation signal works independently of the activity of the enzyme (e.g., nuclease) in the target sequence.

Suitable protein degradation signals may include, e.g., a PEST signal, a destruction box, or another destabilizing peptide.

A “PEST” sequence is a peptide sequence that is rich in proline (P), glutamic acid (E), serine (S), and threonine (T). This sequence has been shown to reduce the intracellular half-life of a protein, i.e., to function as a protein degradation signal. The examples provided herein utilize a PEST sequence of 42 amino acids (SEQ ID NO: 4 KLSHGFPPEVEEQDDGTLPMSCAQESGMDRHPAACASARINV; coding sequence SEQ ID NO: 3). However, in certain embodiments, longer amino acid sequences containing this sequence, or shorter amino acid fragments fragment of this sequence may be selected, e.g., the sequence may be truncated at the amino- and/or carboxy-terminus to be about 10, 15, 20, 25, 30, 35, 40, or 41 amino acids in length.

In certain embodiments, another protein degradation signal sequence may be selected. Such protein degradation signal sequences may be from about 10 amino acids to about 50 amino acids in length, or values therebetween.

In certain embodiment, an ornithine decarboxylase (ODC) degron may be selected as source of suitable protein degradation signal sequence. [J Erales and P. Coffin, Biochimca et Biophsyica Acta, 1843 (2014) 216-221. In another embodiment, a ubiquitin degron may be selected as a protein degradation signal [K T Foitmann, et al, J Mol Biol. 2015 Aug. 28; 427(17): 2748-2756]. Still other degrons may be selected.

A protein degradation signal may be engineered at the amino (N-) terminus of the nuclease coding sequence. Optionally, multiple protein degradation signals may be present. Where two or more protein degradation signals are present, they may be the same or different. Further, where a fusion protein is provided which contains two or more protein degradation signals, one or more of the signals may be located at the N-terminus, one or more of the signals may be located at the carboxy terminus, or combinations thereof (e.g., one signal may be located at the N-terminus and one may be located at the carboxy terminus of a single fusion protein; one signal may be located at the N-terminus and two signals may be located at the carboxy terminus of a single fusion protein, two protein degradation signals may be located at the N-terminus and one signal may be located at the carboxy terminus of a single fusion protein etc.).

The examples herein illustrate use of AAV vectors containing the PEST sequence(s) in the vector genome. In certain embodiments, the vector genome may be packaged into a different vector (e.g., a recombinant bocavirus). In certain embodiments, the expression cassette may be packaged into a different viral vector, into a non-viral vector, and/or into a different delivery system.

As used herein, an “expression cassette” refers to a nucleic acid molecule which comprises coding sequences, promoter, and may include other regulatory sequences therefor, which cassette may be engineered into a genetic element and/or packaged into the capsid of a viral vector (e.g., a viral particle). Typically, such an expression cassette for generating a viral vector contains the sequences described herein flanked by packaging signals of the viral genome and other expression control sequences such as those described herein.

The expression cassette typically contains a promoter sequence as part of the expression control sequences or the regulatory sequences. In one embodiment, a tissue specific promoter may be selected. For example, if a liver-specific promoter is desired, one may select from thyroxin binding globulin (TBG), or other liver-specific promoters [see, e.g., The Liver Specific Gene Promoter Database, Cold Spring Harbor, rulai.schl.edu/LSPD,] such as, e.g., alpha 1 anti-trypsin (A1AT); human albumin (Miyatake et al., J. Virol., 71:5124 32 (1997), humAlb); hepatitis B virus core promoter (Sandig et al., Gene Ther., 3:1002 9 (1996)); TTR minimal enhancer/promoter; alpha-antitrypsin promoter; T7 promoter; and LSP (845 nt)25(requires intron-less scAAV). Other promoters, such as viral promoters, constitutive promoters, regulatable promoters [see, e.g., WO 2011/126808 and WO 2013/049493], or a promoter responsive to physiologic cues may be used may be utilized in the vectors described herein. In some embodiments, the promoter or promoter/enhancer is that shown in any of SEQ ID NO: 11-16.

In addition to a promoter, an expression cassette and/or a vector may contain other appropriate “regulatory element” or “regulatory sequence”, which comprise but not limited to enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. In one embodiment, the intron is that shown in any of SEQ ID NO: 11-16. In one embodiment, the polyA is that shown in any of SEQ ID NO: 11-16.

These control sequences or the regulatory sequences are operably linked to the protein and peptide coding sequences.

### Self-Modulating Nuclease Expression Cassette

In certain embodiments, a self-modulating gene editing nuclease expression cassette is provided which contains a nuclease coding sequence which is operably linked to regulatory sequences which direct expression of the nuclease following delivery to a host cell having a sequence to which the nuclease is targeted and at least one nuclease modulating sequence which is selected from the target sequence for the nuclease or a mutated target sequence which is recognized by the nuclease following its expression. Optionally, the nuclease in these embodiments may be in the form of a fusion protein with a protein degradation signal as described in the preceding section, which is incorporated by reference.

The terms “recognition sequence” or “recognition site” refer to a nucleic acid sequence (e.g., DNA, including, e.g., cDNA), that is bound and cleaved by an endonuclease. For certain meganucleases, a recognition sequence is 22 base pairs in length and comprises a pair of inverted, 9 base pair “half sites” which are separated by four base pairs. In the case of a single-chain meganuclease, the N-terminal domain of the protein contacts a first half-site and the C-terminal domain of the protein contacts a second half-site. However, other nucleases and meganucleases may have shorter or longer recognition sites (e.g., about 10 base pairs to about 40 base pairs) as described herein.

As used herein, the term “target site” or “target sequence” refers to a region of the DNA of a cell comprising a recognition sequence for a nuclease. In certain embodiments, the target site or target sequence is in the chromosomal DNA of the cell.

In certain embodiments, the nuclease modulating sequence has a sequence which is the same (100% identical) to the sequence of the nuclease recognition site in the cell over the full-length of the recognition site in the cell. In certain embodiments, the nuclease modulating sequence is 100% identical to the target sequence, but is shorter in length by up to 5-10% (e.g., for a 22 bp recognition site, about 18-20 bp in length).

In certain embodiments, the nuclease modulating sequence has a sequence which has mutant sequences as compared with the recognition site in the cell. Such sequences are designed to have mismatches in one or more base pairs.

In the examples below, the meganuclease illustrated recognizes a 22 bp sequence and as a result each enzyme modulating sequence selected in these expression cassettes was 22 bp. The mutant target sequences for these 22 bp sequences contained up to 35 to 37% mismatches, i.e., 8 bp which differ from the target sequence. However, other variations will be apparent to one of skill in the art.

Fewer or higher percentages of mismatches may be selected. In certain embodiments, only 1 mismatch is present. In other embodiments, 2 to 12 mismatches are present. In certain embodiments, the mismatches are non-consecutive. In certain embodiments two or three of the mismatches may be consecutive nucleotides. Optionally combinations of a single mismatch and consecutive mismatches (e.g., 2 or 3) may be separated by unmutated sequences in a single mutant target. In certain embodiments, other mismatch sensitive nucleases may recognize shorter or longer sequences, e.g., about 12 base pairs to 40 base pairs in length. Mutant target sequences may have 0.5% to 45% mismatches (i.e., divergent nucleotide sequence) from the enzymes' intended target sequence in the target cell. In certain embodiments, the mutant target sequences are engineered, for example, by using off-target prediction/identification methods (such as GUIDE-Seq or ITR-Seq), and according to their rank (indel % in these sequences, or number of GUIDE-Seq reads, ITR-Seq reads), selecting those that can work at different levels, or even better than the intended target sequence.

In certain embodiments, the enzyme modulating sequence, e.g., a mutant target sequence or a target sequence, may be located downstream of a promoter sequence which directs expression of the enzyme.

In certain embodiments, the enzyme modulating sequence, e.g., a mutant target sequence or a target sequence, may be located downstream of the enzyme coding sequence.

In certain embodiments, the enzyme modulating sequence, e.g., a mutant target sequence or a target sequence, may be located within the nuclease coding sequence.

In certain embodiments, a nuclease expression cassette (or a vector genome containing same) has multiple enzyme modulating sequences, which may be the same or different from each other. The enzyme modulating sequences may be located in tandem, or may be separated from each other by one or more of: a non-coding spacer, an intron, between introns, or another regulatory element, or the enzyme coding sequence. For example, at least a first enzyme modulating sequence may be located upstream of the enzyme coding sequence and a second enzyme modulating sequence may be located downstream of the enzyme coding sequence (e.g., prior to the polyA). Where multiple enzyme modulating sequences are present which are different, at least one is a mutant target sequence. In such embodiments, two or more different mutant target sequences may be engineered in the nuclease expression cassette. In certain embodiments, an enzyme modulating sequence is located within the nuclease coding sequence (e.g., at the 5′ or 3′ end thereof). In one embodiment the target sequence is SEQ ID NO: 5 -TGGACCTCTTTGCCCCAGGGGA. In one embodiment, the mutant target sequence is SEQ ID NO: 6 — TTGCCCTTTTTATTCCCAGGGA.

In one embodiment, a nucleic acid molecule is provided which encodes a fusion protein comprising a PCSK9 meganuclease and a protein degradation signal (e.g., a PEST) sequence. In certain embodiments, a meganuclease may be selected from those described in WO 2018/195449A1.

In one embodiment, a nucleic acid molecule is provided which encodes a fusion protein comprising a TTR meganuclease and a protein degradation signal (e.g., a PEST) sequence. In certain embodiments, the TTR-Target is SEQ ID NO: 7: GCTGGACTGGTATTTGTGTCTG. In certain embodiments, the TTR-MutTarget has the sequence SEQ ID NO: 8: TCGGGACTTTTGTTTGCCTCTT.

In one embodiment, a nucleic acid molecule is provided which encodes a fusion protein comprising a HOA meganuclease and a protein degradation signal (e.g., a PEST) sequence.

In one embodiment, a nucleic acid molecule is provided which encodes a fusion protein comprising a BCKDC meganuclease and a protein degradation signal (e.g., a PEST) sequence.

In one embodiment, a nucleic acid molecule is provided which encodes a fusion protein comprising an APOC3 meganuclease and a protein degradation signal (e.g., a PEST) sequence.

In one embodiment, a nucleic acid molecule is provided which encodes a PCSK9 meganuclease and at least one target sequence or mutant target sequence. In certain embodiments, the meganuclease is expressed as a meganuclease-PEST fusion protein.

In one embodiment, a nucleic acid molecule is provided which encodes a fusion protein comprising a TTR meganuclease and at least one target sequence or mutant target sequence. In certain embodiments, the meganuclease is expressed as a meganuclease-PEST fusion protein.

Optionally, the expression cassette may include miRNA target sequences in the untranslated region(s). The miRNA target sequences are designed to be specifically recognized by miRNA present in cells in which transgene expression is undesirable and/or reduced levels of transgene expression are desired. In certain embodiments, the expression cassette includes miRNA target sequences that specifically reduce expression of the nuclease in dorsal root ganglion. In certain embodiments, the miRNA target sequences are located in the 3′ UTR, 5′ UTR, and/or in both 3′ and 5′ UTR, In some embodiments, the miRNA target sequences are operably linked to the regulatory sequences in the expression cassette. In certain embodiments, the expression cassette comprises at least two tandem repeats of drg-specific miRNA target sequences, wherein the at least two tandem repeats comprise at least a first miRNA target sequence and at least a second miRNA target sequence which may be the same or different. In certain embodiments, the tandem miRNA target sequences are continuous or are separated by a spacer of 1 to 10 nucleic acids, wherein said spacer is not an miRNA target sequence. A discussion of the miRNA DRG-specific sequences is provided in International Patent Application No. PCT/US19/67872, filed Dec. 20, 2019, entitled “Compositions for DRG-Specific Reduction of Transgene Expression”, which document is specifically incorporated herein by reference.

### Viral and Non-Viral Vectors

The expression cassette described herein, containing a nuclease coding sequence and at least one protein degradation signal and/or at least one target or mutant target site, may be engineered into any suitable genetic element for delivery to a target cell.

A “vector” as used herein is a biological or chemical moiety comprising a nucleic acid sequence which can be introduced into an appropriate host cell for replication or expression of said nucleic acid sequence. Common vectors include non-viral vectors and viral vectors. As used herein, a non-viral system might be selected from nanoparticles, electroporation systems and novel biomaterials, naked DNA, phage, transposon, plasmids, cosmids (Phillip McClean, www.ndsu.edu/pubweb/˜mcclean/-plsc731/cloning/cloning4.htm) and artificial chromosomes (Gong, Shiaoching, et al. “A gene expression atlas of the central nervous system based on bacterial artificial chromosomes.” Nature 425.6961 (2003): 917-925).

“Plasmid” or “plasmid vector” generally is designated herein by a lower case p preceded and/or followed by a vector name. Plasmids, other cloning and expression vectors, properties thereof, and constructing/manipulating methods thereof that can be used in accordance with the present invention are readily apparent to those of skill in the art. In one embodiment, the nucleic acid sequence as described herein or the expression cassette as described herein are engineered into a suitable genetic element (a vector) useful for generating viral vectors and/or for delivery to a host cell, e.g., naked DNA, phage, transposon, cosmid, episome, etc., which transfers the nuclease sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

In certain embodiments, the expression cassette is located in a vector genome for packaging into a viral capsid. For example, for an AAV vector genome, the components of the expression cassette are flanked at the extreme 5′ end and the extreme 3′ end by AAV inverted terminal repeat sequences. For example, a 5′ AAV ITR, expression cassette, 3′ AAV ITR. In other embodiments, a self-complementary AAV may be selected. In other embodiments, retroviral system, lentivirus vector system, or an adenoviral system may be used. In one embodiment, the vector genome is that shown in any of SEQ ID NO: 11-16.

In one embodiment, a viral or non-viral vector is provided which comprises nucleic acid molecule which encodes a fusion protein comprising a PCSK9 meganuclease and a PEST sequence. In certain embodiments, a meganuclease may be selected from those described in WO 2018/195449A1.

In one embodiment, a viral or non-viral vector is provided which comprises nucleic acid molecule which encodes a fusion protein comprising a TTR meganuclease and a PEST sequence.

In one embodiment, a viral or non-viral vector is provided which comprises a nucleic acid molecule which encodes a fusion protein comprising a HOA1-2 or HOA3-4 meganuclease and a PEST sequence.

In one embodiment, a viral or non-viral vector is provided which comprises nucleic acid molecule which encodes a fusion protein comprising a BCKDC meganuclease and a protein degradation signal (e.g., a PEST) sequence.

In one embodiment, a viral or non-viral vector is provided which comprises nucleic acid molecule which encodes a fusion protein comprising an APOC3 meganuclease and a protein degradation signal (e.g., a PEST) sequence.

In one embodiment, a viral or non-viral vector is provided which comprises a nucleic acid molecule is provided which encodes a PCSK9 meganuclease and at least one target sequence or mutant target sequence. In certain embodiments, the meganuclease is expressed as a meganuclease-PEST fusion protein.

In one embodiment, a viral or non-viral vector is provided which comprises a nucleic acid molecule which encodes a fusion protein comprising a TTR meganuclease and at least one target sequence or mutant target sequence. In certain embodiments, the meganuclease is expressed as a meganuclease-PEST fusion protein.

In one embodiment, a viral or non-viral vector is provided which comprises a nucleic acid molecule which encodes a fusion protein comprising a HOA1-2 or HOA3-4 meganuclease and at least one target sequence or mutant target sequence. In certain embodiments, the meganuclease is expressed as a meganuclease-PEST fusion protein.

In one embodiment, a viral or non-viral vector is provided which comprises a nucleic acid molecule which encodes a fusion protein comprising a APOC3 meganuclease and at least one target sequence or mutant target sequence. In certain embodiments, the meganuclease is expressed as a meganuclease-PEST fusion protein.

In one embodiment, a viral or non-viral vector is provided which comprises a nucleic acid molecule which encodes a fusion protein comprising a BCKDC meganuclease and at least one target sequence or mutant target sequence. In certain embodiments, the meganuclease is expressed as a meganuclease-PEST fusion protein.

Any suitable non-viral vector (e.g., a plasmid or other genetic element), or a viral vector may be selected. A viral vector may be a recombinant bocavirus, a recombinant lentivirus, a recombinant adenovirus, or a recombinant adeno-associated virus.

### AAV Vectors

In certain embodiments, a recombinant AAV is provided. A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The source of the AAV capsid may be one of any of the dozens of naturally occurring and available adeno-associated viruses, as well as engineered AAVs. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, WO 2003/042397 (rh.10) and WO 2018/160582 (AAVhu68). These documents also describe other AAV which may be selected for generating AAV and are incorporated by reference.

Unless otherwise specified, the AAV capsid, ITRs, and other selected AAV components described herein, may be readily selected from among any AAV, including, without limitation, the AAVs commonly identified as AAV1, AAV2, AAV3, AAV4, AAVS, AAV6, AAV7, AAV8, AAV9, AAV8bp, AAV7M8, AAVAnc80, AAVrh10, and AAVPHP.B and variants of any of the known or mentioned AAVs or AAVs yet to be discovered or variants or mixtures thereof. See, e.g., WO 2005/033321, which is incorporated herein by reference. In one embodiment, the AAV capsid is an AAV1 capsid or variant thereof, AAV8 capsid or variant thereof, an AAV9 capsid or variant thereof, an AAVrh.10 capsid or variant thereof, an AAVrh64R1 capsid or variant thereof, an AAVhu.37 capsid or variant thereof, or an AAV3B or variant thereof. See, also PCT/US19/19804 and PCT/US19/19861, each entitled “NOVEL ADENO-ASSOCIATED VIRUS (AAV) VECTORS, AAV VECTORS HAVING REDUCED CAPSID DEAMIDATION AND USES THEREFOR” and filed Feb. 27, 2019, which are incorporated by reference herein in their entirety.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein.

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016 and its priority documents, U.S. Patent Application No. 62/322,071, filed Apr. 13, 2016 and U.S. Patent Application No. 62/226,357, filed Dec. 11, 2015 and entitled “Scalable Purification Method for AAV9”, which is incorporated by reference herein. Purification methods for AAV8, International Patent Application No. PCT/US2016/065976, filed Dec. 9, 2016 and its priority documents U.S. Patent Application No. 62/322,098, filed Apr. 13, 2016 and U.S. Patent Application No. 62/266,341, filed Dec. 11, 2015, and rh10, International Patent Application No. PCT/US16/66013, filed Dec. 9, 2016 and its priority documents, U.S. Patent Application No. 62/322,055, filed Apr. 13, 2016 and U.S. Patent Application No. 62/266,347, entitled “Scalable Purification Method for AAVrh10”, also filed Dec. 11, 2015, and for AAV1, International Patent Application No. PCT/US2016/065974, filed Dec. 9, 2016 and its priority documents U.S. Patent Application Nos. 62/322,083, filed Apr. 13, 2016 and 62/26,351, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL—GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J Vivol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, California) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April;25(2):115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1)]which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Pharmaceutical Compositions

A pharmaceutical composition comprises one or more of an expression cassette, vector containing same (viral or non-viral) or another system containing the expression cassette and one or more of a carrier, suspending agent, and/or excipient.

In certain embodiments, compositions containing at least one rAAV stock (e.g., an rAAV stock) and an optional carrier, excipient and/or preservative. An rAAV stock refers to a plurality of rAAV vectors which are the same, e.g., such as in the amounts described below in the discussion of concentrations and dosage units. The rAAV vector delivered transgenes may be formulated for delivery either encapsulated in a lipid particle, a liposome, a vesicle, a nanosphere, or a nanoparticle or the like.

In certain embodiments, an expression cassette is delivered via a lipid nanoparticle. The term “lipid nanoparticle” refers to a lipid composition having a typically spherical structure with an average diameter of 10 to 1000 nanometers, e.g. 75 nm to 750 nm, or 100 nm and 350 nm, or between 250 nm to about 500 nm. In some formulations, lipid nanoparticles can comprise at least one cationic lipid, at least one noncationic lipid, and at least one conjugated lipid. Lipid nanoparticles known in the art that are suitable for encapsulating nucleic acids, such as mRNA, may be used. “Average diameter” is the average size of the population of nanoparticles comprising the lipophilic phase and the hydrophilic phase. The mean size of these systems can be measured by standard methods known by the person skilled in the art. Examples of suitable lipid nanoparticles for gene therapy is described, e.g., L. Battaglia and E. Ugazio, J Nanomaterials, Vol 2019, Article ID 283441, pp. 1-22; US2012/0183589A1; and WO 2012/170930 which are incorporated herein by reference in their entirety.

In certain embodiments, a composition is provided which comprises a nucleic acid molecule encoding a nuclease—degradation peptide signal fusion protein and a pharmaceutically acceptable diluent, carrier, and/or excipient.

An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney,), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10″ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10⁵, 8×10¹⁵, or 9×10¹⁵ GC per dose including all integers or fractional amounts within the range. In one embodiment, for human application the dose can range from 1×10¹° to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method.

Any suitable route of administration may be selected (e.g., oral, inhalation, intranasal, intratracheal, intraarterial, intraocular, intravenous, intramuscular, intraperitoneal, and other parental routes). Accordingly, pharmaceutical compositions may be formulated for any appropriate route of administration, for example, in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

### Methods

The compositions provided herein are useful for reducing off-target activity of enzymes delivered in vivo. In certain embodiments, the compositions are useful in reducing off-target activity of an enzyme expressed following non-viral mediated delivery of an expression cassette comprising the enzyme coding sequence, an enzyme-PEST coding sequence, and/or an enzyme-PEST coding sequence with one or more enzyme modulating (target or mutant target) sequences. In certain embodiments, the compositions are useful in reducing off-target activity of an enzyme expressed following AAV-mediated delivery of a vector genome.

In certain embodiments, the effectiveness of a protein degradation signal (e.g., a PEST, Box, or other degron) may be assessed in vitro. For example, the half-life of a fusion protein containing an enzyme (e.g., nuclease) and a protein degradation signal may be assessed in vitro (in cultured cells) by treating the cells to stop translation of the protein (e.g., with cycloheximide (CHX)) and then performing a western blot at different times post-treatment. Other suitable methods for assessing degradation of a nuclease may be readily determined by one of skill in the art.

A reduction in off-target nuclease activity (or an increase in nuclease specificity) can be determined using a variety of approaches which have been described in the literature. Such methods for determining nuclease specificity, include cell-free methods such as Site-Seq [Cameron, P., et al, (2017) Mapping the genomic landscape of CRISPR-Cas9 cleavage. Nat Methods, 14, 600-606], Digenome-seq [Kim, D., et al, (2015) Digenome-seq: genome-wide profiling of CRISPR-Cas9 off-target effects in human cells. Nat Methods, 12, 237-243, 231 p following 243], and Circle-Seq [Tsai, S.Q., et al, (2017) CIRCLE-seq: a highly sensitive in vitro screen for genome-wide CRISPR-Cas9 nuclease off-targets. Nat Methods, 14, 607-614] and in vitro-based methods such as, e.g., GUIDE-Seq [Tsai (2017) Nat Methods, 14, 607-614] and Integrative-Deficient Lentiviral Vectors Capture (IDLV) [Gabriel, R., et al. (2011) An unbiased genome-wide analysis of zinc-finger nuclease specificity. Nat Biotechnol, 29, 816-823;Wang, X., et al., (2015) Unbiased detection of off-target cleavage by CRISPR-Cas9 and TALENs using integrase-defective lentiviral vectors. Nat Biotechnol, 33, 175-178].

Provided herein and incorporated by reference is an improved assay, which more accurately predicts the number and rate of off-target activity in vivo.

The ITR-seq method we developed provides an unbiased, genome-wide identification of sites of ITR integration. The example below uses the AAV ITR as a tag for identifying DSB, we can measure the off-target activity of genome-editing nucleases in vivo. However, it will be readily understood that any terminal repeat sequence (trs) (e.g., a lentivirus terminal repeat) or other common integration site, e.g., a repeat sequence which is located 5′ and 3′ of the expression cassette, may be used. Similarly, a non-viral expression cassette may be engineered to contain such a common integration site in order to allow detection using this assay (e.g., a trs may be engineered into a DNA expression cassette which is delivered by a non-viral or non-vector delivery system).

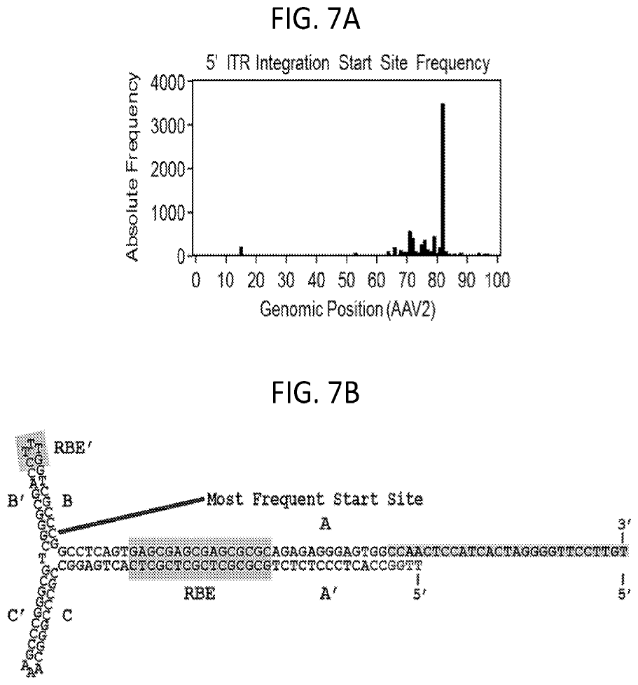

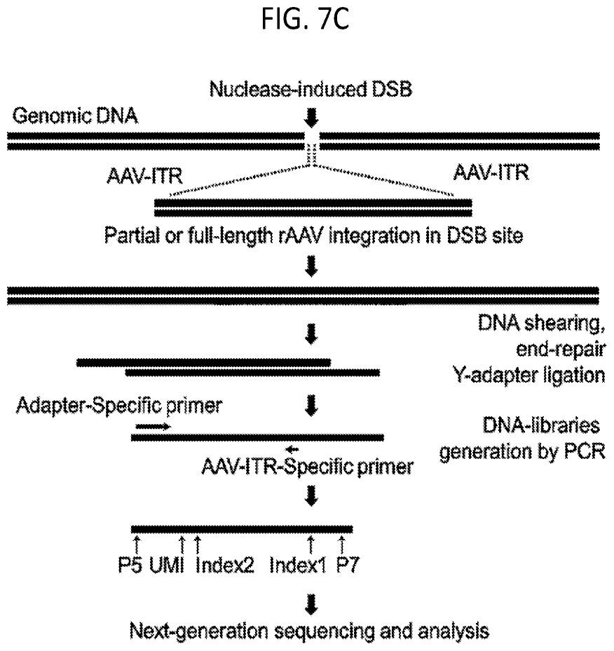

The ITR-Seq protocol is a modified version of an anchored PCR reaction, in which a single primer is designed to anneal to and amplify outward from the ITR sequence (e.g., FIG. 7C). Following ITR integration in the DNA, the primer may be used to amplify the junction of the host genome and the inserted vector ITR sequence (e.g., FIGS. 7B, 7C). In order to adequately denature the ordered secondary structure of the integrated ITR, a high annealing temperature is used (e.g., 69° C.) and longer adapter-specific primers. In certain embodiments, the “high annealing temperature” may be any temperature at which the PCR polymerase functions and the primers anneal to the target sequence, e.g., at 60° C. to 75° C., or about 68° C. to 72° C. In certain embodiments, the primer sequence may be from 18 to about 42 nucleotides in length, or longer if specificity is retained. In certain embodiments, the primer is at least 20 nucleotides to 40 nucleotides, at least 30 nucleotides to 40 nucleotides, at least 35 nucleotides to 40 nucleotides, or about 37 nucleotides in length.

To use the ITR-Seq assay for sample analysis, DNA is isolated from a sample (e.g., from the tissues of animals treated with nuclease-expressing AAV vectors). The DNA is sheared and ligated it to Y-adapters, as described in previous reports [Tsai, S.Q., et al, (2015) GUIDE-seq enables genome-wide profiling of off-target cleavage by CRISPR-Cas nucleases. Nat Biotechnol, 33, 187-197]. Following two rounds of PCR using the ITR-specific primer described above and adapter-specific primers, NGS-compatible libraries are produced. After sequencing, the resulting amplicons are determined computationally that contain both the amplified ITR sequence and adjacent genomic DNA sequences. The location and frequency of genome-wide ITR integration sites is also determined. By requiring that the ITR integrates in both the forward and reverse strand orientations, we further reduced the number of false positives and identify high-confidence ITR-integration sites. For each sample, a rank-ordered list of nuclease target sites (ITR-Seq rank) and sorted the sites in decreasing order by the total number of observed ITR-integration events per locus (ITR-Seq reads) are produced. ITR-Seq reports are generated at the end of the computational analysis with the most probable off-target sequence (based on the homology to the intended target sequence), the genomic location, and the ITR-Seq rank (according to the number of NGS reads mapping to the corresponding locus). Example 3 provides additional details of the assay and illustrates the use of the assay.

In one aspect, a method for editing of a targeted gene is provided which comprises delivering a self-modulating nuclease expression cassette as described herein.

In one aspect, a method for editing of a targeted gene is provided which comprises delivering a composition as described herein.

In one aspect, a method for editing of a targeted gene is provided which comprises delivering a viral or non-viral vector as described herein.

In one aspect, a method for editing of a targeted gene is provided which comprises delivering an rAAV as described herein.

In one aspect, a method for treating a patient having cholesterol-related disorders, such as hypercholesterolemia, using a self-modulating nuclease expression cassette comprising meganuclease which recognizes a site within the human PCSK9 gene as described herein. In certain embodiments, the expression cassette encodes a fusion protein comprising a PCSK9 meganuclease and a protein degradation signal, e.g., a PEST sequence. In one embodiment, the fusion protein has the sequence shown in SEQ ID NO: 18. In certain embodiments, the expression cassette encodes a PCSK9 meganuclease and at least one target sequence. In certain embodiments, the expression cassette comprises a mutant target sequence. In certain embodiments, the expression cassette comprises the fusion protein comprising a PCSK9 meganuclease and a protein degradation signal and at least one target sequence. Such expression cassettes may be delivered via a viral or non-viral vector. In certain embodiments, the expression cassettes may be delivered using an LNP.

In one aspect, provided is a method for treating a patient having a disorder associated with a defect in the alanine glyoxylate aminotransferase gene, such as primary hyperoxaluria type 1, using a self-modulating nuclease expression cassette comprising a meganuclease which recognizes a site within the human HAO gene as described herein. In certain embodiments, the expression cassette encodes a fusion protein comprising a HOA meganuclease and a protein degradation signal, e.g., a PEST sequence. In certain embodiments, the expression cassette encodes a HOA meganuclease and at least one target sequence. In certain embodiments, the expression cassette comprises a mutant target sequence. In certain embodiments, the expression cassette comprises the fusion protein comprising a HOA meganuclease and a protein degradation signal and at least one target sequence. Such expression cassettes may be delivered via a viral or non-viral vector. In certain embodiments, the expression cassettes may be delivered using an LNP. In certain embodiments, the disorder is primary hyperoxaluria (PH1).

In one aspect, a method for treating a patient having a disorder associated with a defect in the transthyretin (TTR) gene is provided, using a self-modulating nuclease expression cassette comprising a meganuclease which recognizes a site within the human TTR gene as described herein. In certain embodiments, the expression cassette encodes a fusion protein comprising a TTR meganuclease and a protein degradation signal, e.g., a PEST sequence. In certain embodiments, the expression cassette encodes a TTR meganuclease and at least one target sequence. In certain embodiments, the expression cassette comprises a mutant target sequence. In certain embodiments, the expression cassette comprises the fusion protein comprising a TTR meganuclease and a protein degradation signal and at least one target sequence. Such expression cassettes may be delivered via a viral or non-viral vector. In certain embodiments, the expression cassettes may be delivered using an LNP. In certain embodiments, the disorder is TTR-related hereditary amyloidosis.

In another aspect, a method for treating a patient having a disorder associated with a defect in the apoliprotein C-II (APOC3) gene is provided, using a self-modulating nuclease expression cassette comprising a meganuclease which recognizes a site within the human APOC3 gene as described herein. In certain embodiments, the expression cassette encodes a fusion protein comprising an APOC3 meganuclease and a protein degradation signal, e.g., a PEST sequence. In certain embodiments, the expression cassette encodes an APOC3 meganuclease and at least one target sequence. In certain embodiments, the expression cassette comprises a mutant target sequence. In certain embodiments, the expression cassette comprises the fusion protein comprising an APOC3 meganuclease and a protein degradation signal and at least one target sequence. Such expression cassettes may be delivered via a viral or non-viral vector. In certain embodiments, the expression cassettes may be delivered using an LNP.

In one aspect, a method for treating a patient having a disorder associated with a defect in the branched-chain a-ketoacid dehydrogenase complex (BCKDC) E1 α gene is provided, using a self-modulating nuclease expression cassette comprising a meganuclease which recognizes a site within the human BCKDC E1 α gene. In certain embodiments, the expression cassette encodes a fusion protein comprising a BCKDC meganuclease and a protein degradation signal, e.g., a PEST sequence. See, WO 2020/056155A2. In certain embodiments, the expression cassette encodes a BCKDC meganuclease and at least one target sequence. In certain embodiments, the expression cassette comprises a mutant target sequence. In certain embodiments, the expression cassette comprises the fusion protein comprising a BCKDC meganuclease and a protein degradation signal and at least one target sequence. Such expression cassettes may be delivered via a viral or non-viral vector. In certain embodiments, the expression cassettes may be delivered using an LNP. In certain embodiments, the disorder is maple syrup urine disease.

In certain embodiments, nucleases other than meganucleases targeting any of the above-described genes are contemplated.

In certain embodiments, a nuclease expression cassette, non-viral vector, viral vector (e.g.,rAAV), as described herein is administrable for gene editing in a patient. In certain embodiments, the method is useful for non-embryonic gene editing. In certain embodiments, the patient is an infant (e.g., birth to about 9 months). In certain embodiments, the patient is older than an infant, e.g, 12 months or older.

In certain embodiments, a pharmaceutical composition as described herein is administrable for gene editing in a patient. In certain embodiments, the method is useful for non-embryonic gene editing. In certain embodiments, the patient is an infant (e.g., birth to about 9 months). In certain embodiments, the patient is older than an infant, e.g, 12 months or older.

As used herein, “a,” “an,” or “the” can mean one or more than one. For example, “a” cell can mean a single cell or a multiplicity of cells.

In certain embodiments, the term “meganuclease” refers to an endonuclease that binds double-stranded DNA at a recognition sequence that is greater than 12 base pairs. Preferably, the recognition sequence for a meganuclease of the invention is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art. See, e.g., WO 2007/047859). A meganuclease as used herein binds to double-stranded DNA as a heterodimer. A meganuclease may also be a “single-chain meganuclease” in which a pair of DNA-binding domains are joined into a single polypeptide using a peptide linker. The term “homing endonuclease” is synonymous with the term “meganuclease.” See, WO 2018/195449, describing certain PCSK9 meganucleases, which is incorporated herein in its entirety.

As used herein, the term “specificity” means the ability of a meganuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific meganuclease is capable of cleaving only one or a very few recognition sequences. Specificity can be determined by any method known in the art.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same expression cassette or host cell, but which is present in a non-natural state, e.g. a different copy number, or under the control of different regulatory elements.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene.

As used herein, the term “host cell” may refer to the packaging cell line in which a vector (e.g., a recombinant AAV) is produced from a production plasmid. In the alternative, the term “host cell” may refer to any target cell in which expression of the transgene is desired. Thus, a “host cell,” refers to a prokaryotic or eukaryotic cell that contains a exogenous or heterologous nucleic acid sequence that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. Still in other embodiment, the term “host cell” is intended to reference the target cells of the subject being treated in vivo for the diseases or conditions as described herein. In certain embodiments, the term “host cell” is a liver cell or hepatocyte.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and

“MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the term “about” refers to a variant of ±10% from the reference integer and values therebetween. For example “about” 40 base pairs, includes ±4 (i.e., 36-44, which includes the integers 36, 37, 38, 39, 40, 41, 42, 43, 44). For other values, particularly when reference is to a percentage (e.g., 90% identity, about 10% variance, or about 36% mismatches), the term “about” is inclusive of all values within the range including both the integer and fractions.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

We hypothesized that low intracellular levels of a meganuclease are sufficient for on-target genome editing, and that surpassing this threshold increases the chance of off-target editing. To test this hypothesis, we developed a self-inactivation or “suicide” system to restrict expression of the meganuclease.

In conclusion, restricting meganuclease expression through self-inactivation by inserting the target sequence and/or adding a protein degradation signal reduces off-target activity without compromising on-target efficacy. Including this suicide system in gene editing approaches increases the safety profile of AAV-delivered genome editing nucleases.

### Example 1

In this example, engineered meganucleases described in WO2018/195449 were used to illustrate the invention. These meganucleases were engineered to recognize and cleave the PCS 7-8 recognition sequence. The PCS 7-8 recognition sequence is positioned within the PCSK9 gene. These engineered meganucleases comprise a first subunit, comprising a first hypervariable (HVR1) region, and a second subunit, comprising a second hypervariable (HVR2) region. Further, the first subunit binds to a first recognition half-site in the recognition sequence (e.g., the PCS7 half-site), and the second subunit binds to a second recognition half-site in the recognition sequence (e.g., the PCS7 half-site). In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit. See, e.g, Table 1 of WO 2018/195449.

We developed AAV vectors expressing M2PCSK9 by inserting the 22 bp meganuclease target sequence after the promoter. With this design, expressed M2PCSK9 should both edit the PCSK9 gene and cleave the AAV vector genome immediately after the promoter, preventing further transcription of the meganuclease transgene. We constructed alternative vectors by inserting an additional target sequence before the polyA sequence or by inserting a mutant target sequence after the promoter. We also included the PEST sequence in frame with the M2PCSK9 meganuclease, as this sequence should target the transgene protein for degradation by proteasomes.

We intravenously administered immunodeficient RAG knockout mice with an AAV9 vector expressing human PCSK9. Two weeks later, we readministered the mice with the suicide system vectors. All versions of the suicide system reduced the levels of PCSK9 in serum, albeit to different degrees and with different time courses post-vector administration. As expected, M2PCSK9 created indels in both the target sequence in the PSCK9 gene as well as at the target sequence when present in the AAV genome. For some of the suicide system vectors, the on-target editing efficacy was comparable to that obtained with the parental AAV-M2PCSK9 vector with a reduction in protein expression determined by Western blot and a 20-fold reduction in off-target activity at 9 weeks post-vector administration.

Plasmids

All the constructs are based on a AAV plasmid containing : AAV inverted terminal repeats (ITR), human thyroid hormone-binding globulin (TBG) promoter, Promega intron, the PCS7-8L.197 (also known as ARCUS2 or M2PCSK9) gene, Woodchuck Hepatitis Virus (WHP) Posttranscriptional Regulatory Element (WPRE), the bovine growth hormone (bGH) polyA signal, and a second AAV ITR sequence (this plasmid has been described in the previous publication (Nat Biotechnol. 2018 September;36(8):717-725).

Plasmids used for AAV production:
pAAV.TBG.PI.PCS 7-8L.197.bGH (AAV.M2PCSK9): WPRE sequence was removed from a plasmid previously described (Nat Biotechnol. 2018 Sep;36(8):717-725).
   Final plasmid contains the TBG promoter, a synthetic intron, the coding sequence for M2PCSK9 (I-Cre-I engineered Meganuclease), and the bovine growth hormone polyadenylation sequence. The sequence of the expression cassette from this plasmid is shown in SEQ ID NO: 9. The amino acid sequence of M2PCSK9 is shown in SEQ ID NO: 10.
pAAV.TBG.AP-TO.PI.PCS 7-8L.197.bGH (AAV.Target.M2PCSK9): The M2PCSK9 target sequence (SEQ ID NO: 5: 5′-TGGACCTCTTTGCCCCAGGGGA-3′) was cloned after the promoter sequence. The sequence of the expression cassette from this plasmid is shown in SEQ ID NO: 11.
pAAV.TBG.AP-TO.PI.PCS 7-8L.197-PEST.bGH (AAV.Target.M2PCSK9+PEST): Vector containing the target sequence as above, and the proline-glutamate-serine-threonine-rich (PEST) sequence from mouse Ornithine decarboxylase, cloned in frame with M2PCSK9 coding sequence. The sequence of the expression cassette from this plasmid is shown in SEQ ID NO: 12.
pAAV.TBG.AP-TO.PI.PCS 7-8L.197-PEST.AP-TO.bGH (AAV.2xTarget.M2PCSK9+PEST): Similar to the previous vector (containing a Target sequence after the promoter and the PEST sequence in frame with M2PCSK9) plus an additional M2PCSK9 target sequence cloned before the polyA signal. The sequence of the expression cassette from this plasmid is shown in SEQ ID NO: 13.
pAAV.TBG.AP-T8VL.PI.PCS 7-8L.197-PEST.bGH (AAV.MutTarget.M2PCSK9+PEST): A Mutant target sequence (5′-TTGCCCTTTTTATTCCCAGGGA-3′) was cloned immediately after the promoter (similar to the AAV8.Target.M2PCSK9+PEST construct), replacing the parental target sequence (5′-TGGACCTCTTTGCCCCAGGGGA-3′). The sequence of the expression cassette from this plasmid is shown in SEQ ID NO: 14.
pAAV.TBG.PI.PCS 7-8L.197-PEST.bGH (AAV.M2PCSK9+PEST): PEST sequence was cloned in frame with M2PCSK9 coding sequence. The sequence of the expression cassette from this plasmid is shown in SEQ ID NO: 15.
pAAV.TBG.AP-T8VL.PI.PCS 7-8L.197.bGH (AAV.MutTarget.M2PCSK9): Mutant target sequence was cloned immediately after the promoter. The sequence of the expression cassette from this plasmid is shown in SEQ ID NO: 16.

To construct and produce the AAV suicide vectors the WPRE element was eliminated. The PCS7-8L.197 target sequence (SEQ ID NO: 5-TGGACCTCTTTGCCCCAGGGGA) was cloned after the TBG promoter and before the Promega intron element. An additional target sequence was cloned after the M2PCSK9 gene and before the polyA signal. A MutTarget sequence (SEQ ID NO: 6: TTGCCCTTTTTATTCCCAGGGA) was identified in GUIDE-Seq experiments in LLC-MK2 cells (Nat Biotechnol. 2018 Sep;36(8):717-725) as a low rank off-target sequence for M2PCSK9.

Ornithine decarboxylase (ODC) proline-glutamate-serine-threonine-rich (PEST) sequence: SEQ ID NO: 3: aagcttagcc atggcttccc gccggaggtg gaggagcagg atgatggcac gctgcccatg tcttgtgccc aggagagcgg gatggaccgt caccctgcag cctgtgcttc tgctaggatca atgtgtagtaa) encodes for the amino acid sequence: KLSHGFPPEVEEQDDGTLPMSCAQESGMDRHPAACASARINV (SEQ ID NO: 4). This PEST sequence was obtained from mouse cells and cloned in-frame with the M2PCSK9 gene sequence in the vectors.

AAV vectors were produced using triple transfection techniques as previously described.

Mouse Experiments

3.5×10¹⁰ genome copies (GC) of AAV serotype 9 encoding for the human proprotein convertase subtilisin/kexin type 9 enzyme (AAV9.hPCSK9) were administered to Male 6- to 8-week-old Ragl KO mice (The Jackson Laboratory) via a single vein tail injection. Two weeks later, 1×10¹¹ or 1×10¹² GC of AAV serotype 8 encoding for the M2PCSK9 nuclease or AAV suicide vectors were injected through a single vein tail injection. Serum samples were collected weekly until the end of the study. A subset of mice were euthanized and liver collected at 4 or 9 weeks post-AAV9.hPCSK9.

Non-Human Primates (NHP) Experiments

6×10¹² GC/Kg of AAV.M2PCSK9, AAV.Target.M2PCSK9, and AAV.MutTarget.M2PCSK9-PEST or 3×10¹³ GC/Kg of AAV.MutTarget.M2PCSK9 were intravenously administered to rhesus macaques. Peripheral blood mononuclear cells (PBMCs) and serum samples were obtained before and at different times after vector administration. A liver biopsy was collected at day 18 post-vector administration. All the blood tests, including hPCSK9 measurements, were performed as previously described (Nat Biotechnol. 2018 Sep;36(8):717-725).

Indel Analysis

Indels in the target region present in the AAV9.hPCSK9 and in AAV suicide vectors in mouse experiments, as well as in the PCSK9 gene, AAV.Target.M2PCSK9 and AAV.MutTarget.M2PCSK9-PEST vector were quantified as previously described (Nat Biotechnol. 2018 Sep;36(8):717-725). Primers used for this assay are shown in Table 1.

| | | | SEQ ID |
|---|---|---|---|
| Name | Sequence | Region of interest | NO: |
| Target_5F | tgctttgttcctct | 5′ target sequence | 19 |
| | tggccttgg | in AAV vectors | |
| Target_5R | tcctggcaaagatg | | 20 |
| | gaaccgtc | | |
| Target_3F | gactccaagacccg | 3′ target sequence | 21 |
| | caagaccacttc | in AAV vectors | |
| Target_3R | ttattaggaaagga | | 22 |
| | cagtgggagtggca | | |
| | cc | | |
| PCS_MMF | tgccacctacctcc | On-target region | 23 |
| | tcacctttc | of rhesus macaques | |
| | | PCSK9 gene | |
| PCS_MMR | ggctgttagcatca | | 24 |
| | cggtgg | | |
| AAV_PCS_F | cctcagctcccgag | On-target region | 25 |
| | gtcatcacagttg | of AAV9.hPCSK9 | |
| | | vector | |
| AAV_PCS_R | tgacatctttggca | | 26 |
| | gagaagtggatcag | | |
| | tc | | |

AMP-Seq Analysis

Indels and ITR integration in the PCSK9 target region in NHP experiments were determined by AMP-Seq analysis as previously described (Nat Biotechnol. 2018 September;36(8):717-725).

Off-Targets Identification and Characterization

ITR-Seq was performed in liver from mouse and NHP as described in Example 3 below.

Primers for the indicated off-target locations were designed for indel % calculations as previously described (Nat Biotechnol. 2018 September;36(8):717-725).

Results

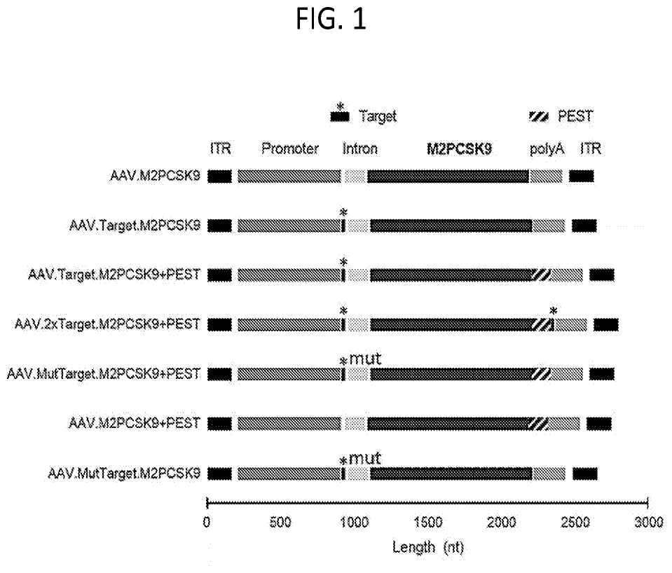

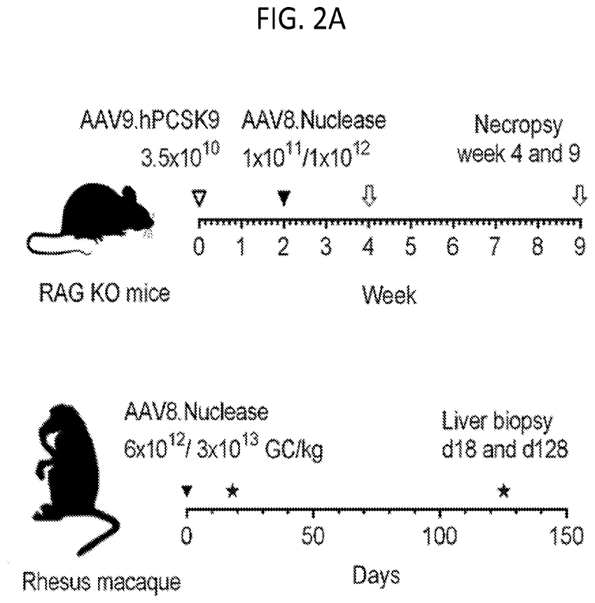

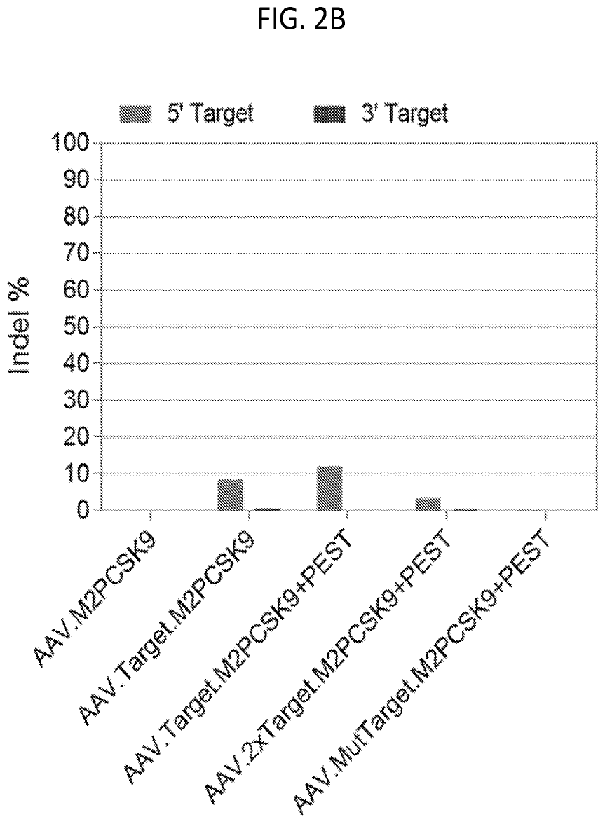

We evaluated if a mutant target sequence, a PEST signal or a combination of these elements in the M2PCSK9-expressing AAV vectors result in a decrease in the off-target activity of the nuclease without compromising its on-target activity. FIG. 1 shows the schematic representation of the tested AAV vectors. FIG. 2A show the timeline of the mouse and NHP studies described herein. FIG. 2B shows the low editing in the AAV genome occurred during the AAV production of the suicide vectors.

To test the in vivo efficacy of these vectors, Ragl KO mice were first injected with an AAV vector expressing hPCSK9 (as the mouse genome does not contain the M2PCSK9 target sequence), two weeks later these mice were administered with AAV suicide vectors at a 1×10¹¹ GC/mouse. Two weeks later (4 weeks since the first vector injection) or seven weeks later (9 weeks in total) mice were euthanized and liver was collected.

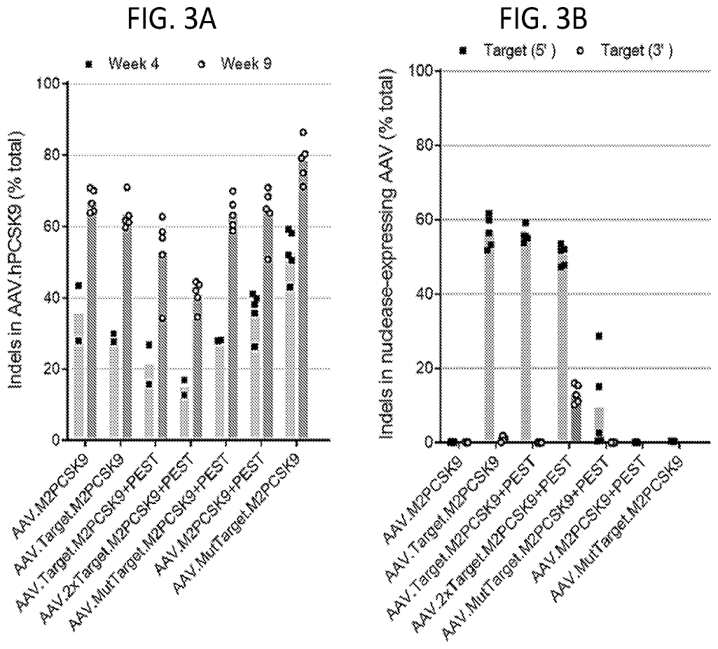

A region encompassing the M2PCSK9 target sequence in the AAV.hPCSK9 vector was amplified by PCR, amplicons were analyzed by next generation sequencing and bioinformatics analysis to determine the percentage of AAV.hPCSK9-derived amplicons containing insertions or deletions (indels) in the target area (FIG. 3A). All the tested AAV suicide vectors induced indels in the AAV.hPCSK9 locus at week 4 and week 9. Additionally, we evaluated if the M2PCSK9 nuclease also induced indels in the target or mutant target sequences present in the AAV vectors expressing the M2PCSK9 nuclease. We found evidence of indels in the target region of all the vectors containing the Target sequence. There were indels in the vector containing both the Mutant Target and PEST sequences and a lower percentage in the vectors containing the mutant target sequence but no the PEST sequence (FIG. 3B).

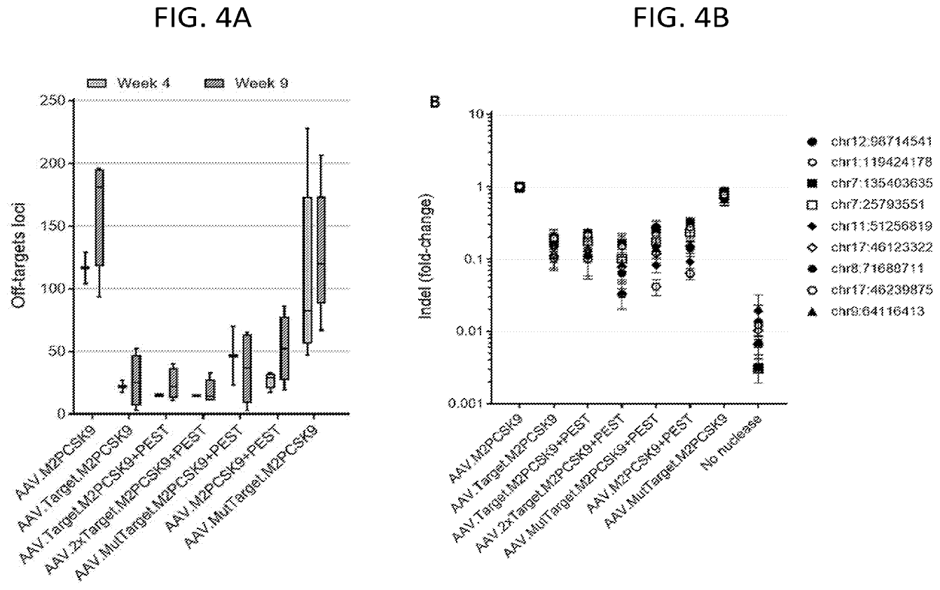

After assessing that the AAV suicide vectors retained on-target activity we determined the off-target activity of M2PCSK9 when its expression was mediated by these vectors. We used a technique called ITR-Seq to determine genomic of double-stranded breaks derived from the on- and off-target activity of the M2PCSK9 nuclease (BMC Genomics. 2020 Mar. 17; 21(1):239). ITR-Seq analysis of liver DNA (FIG. 4A) identified approximately 160 off-target sites in mice treated with AAV.M2PCSK9, while for AAV suicide constructs the number of off-targets was reduced to 26, except for AAV.M2PCKS9+PEST (52 off-targets in average) and AAV.MutTarget.M2PCSK9 (128 off-targets).

From the identified off-targets we selected a subset of high-ranking off-targets for further analysis. We designed primers specific to amplify a DNA region encompassing these off-targets and the indel% was calculated (FIG. 4B). There was a reduction in the indel % in the off-target sites in mice treated with the AAV suicide vectors compared to mice treated with AAV.M2PCSK9 vector. Similar to what was observed in the number of identified off-targets, the indel % in the selected off-targets was similar for mice treated either AAV.M2PCSK9 and AAV.MutTarget.M2PCSK9 (FIG. 4B), suggesting that the mutant target sequence by itself is not enough to mediate a reduction in M2PCSK9 off-target activity.

For further testing in non-human primates (NHP), besides the parental AAV M2PCSK9 vector, we selected two AAV suicide vectors with high on-target activity and a reduced off-target activity: AAV.Target.M2PCSK9 and AAV.MutTarget.M2PCSK9+PEST. The schematic representation of these vectors is shown in FIG. 1.

A 6×10¹² GC/kg dose of each AAV, and a higher dose (3×10¹³ GC/kg) for AAV.MutTarget.M2PCSK9+PEST, were intravenously administered to NHP. A day 18 and day 128 liver biopsies were collected for the treated NHPs. The study is ongoing for some NHP and therefore the day 128 biopsy is yet to be collected.

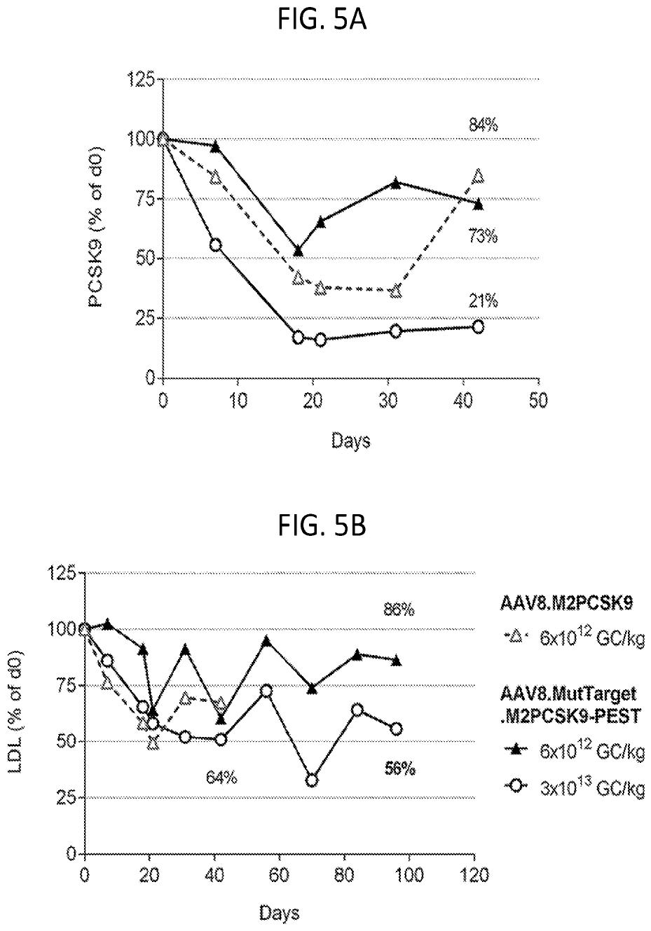

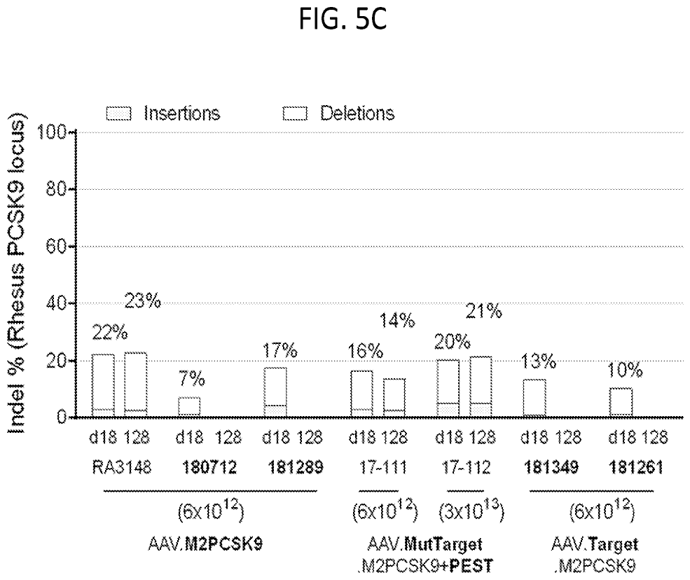

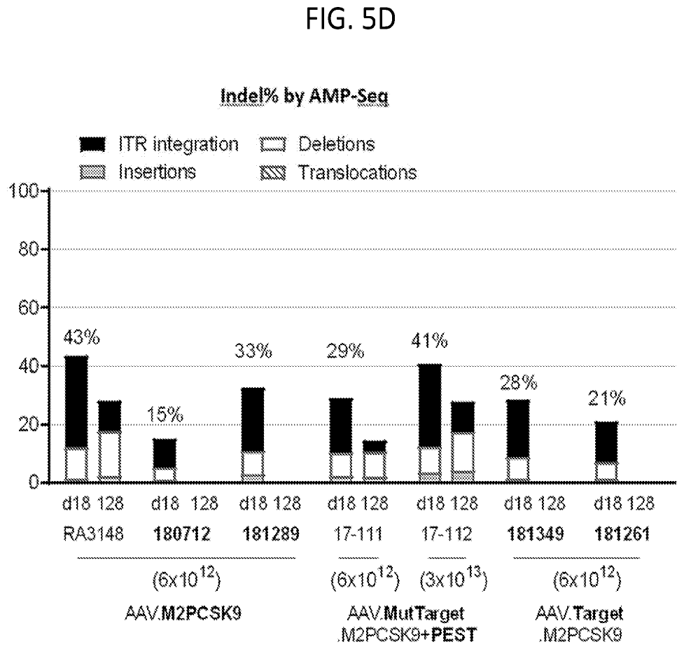

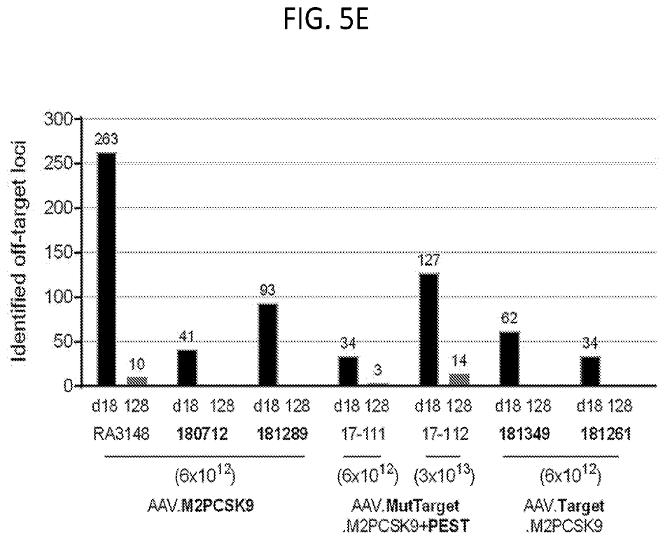

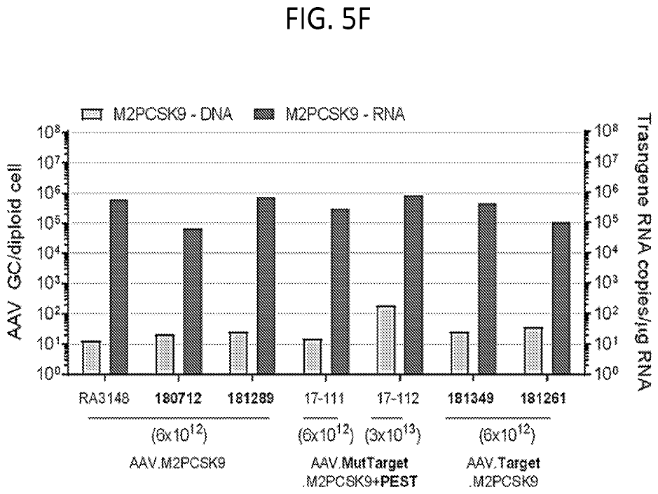

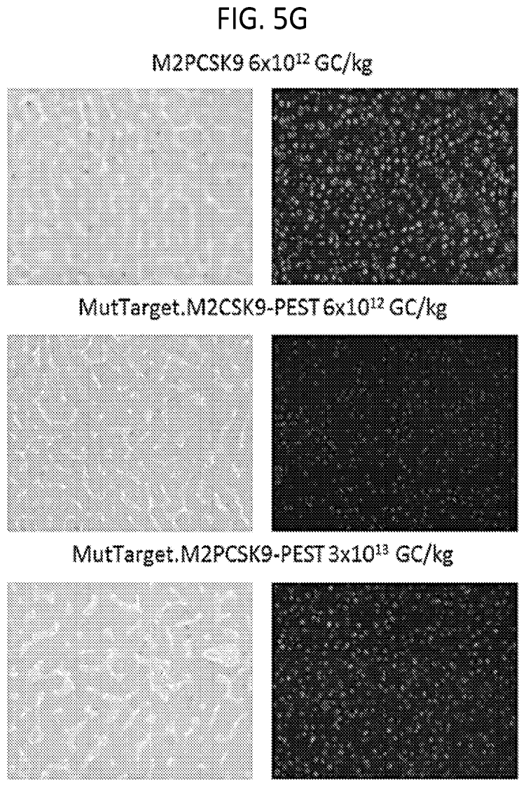

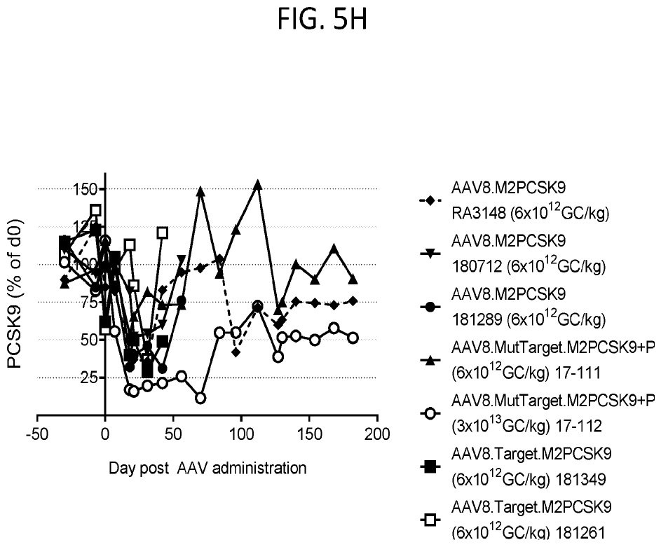

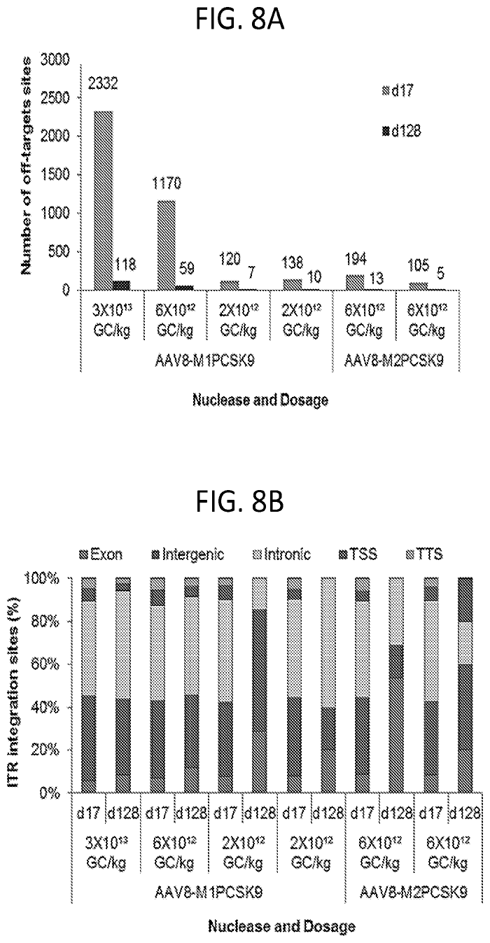

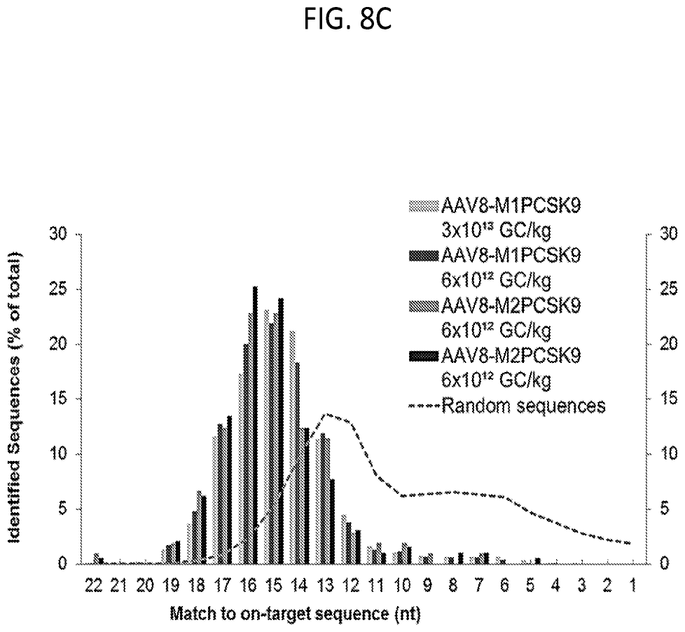

All the tested AAV vectors induced indels in the intended target region in the PCSK9 gene, as detected by Amplicon-Seq or AMP-Seq methods (FIG. 5C and FIG. 5D, respectively). The editing in the PCSK9 target region induced a decrease in PCSK9 as previously observed (Nat Biotechnol. 2018 September;36(8):717-725), which also lead to a reduction in the LDL levels at different times post vector injection (FIG. 7 and FIG. 8, respectively).

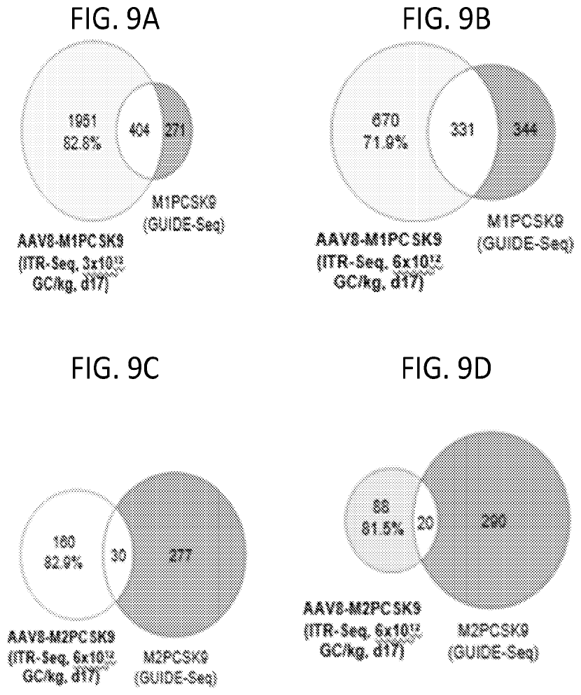

As it possible that editing in the target sequence present in the AAV suicide vectors promotes degradation of the AAV vectors, leading to a subsequent reduction in the transgene RNA levels, we quantified AAV genome copies (GC) and the M2CPSK9 RNA in the liver biopsy samples at d18 (FIG. 9). As expected, AAV GC were higher in the NHP treated with a 3×10¹³ GC/kg dose of AAV.MutTarget+PEST than the rest of the NHP treated with 6×10¹² GC/kg; the amount of GC was similar in the NHPs treated with the same dose. The amount of RNA was similar between all the tested doses, even in the NHP treated with 3×10¹³ GC/kg.

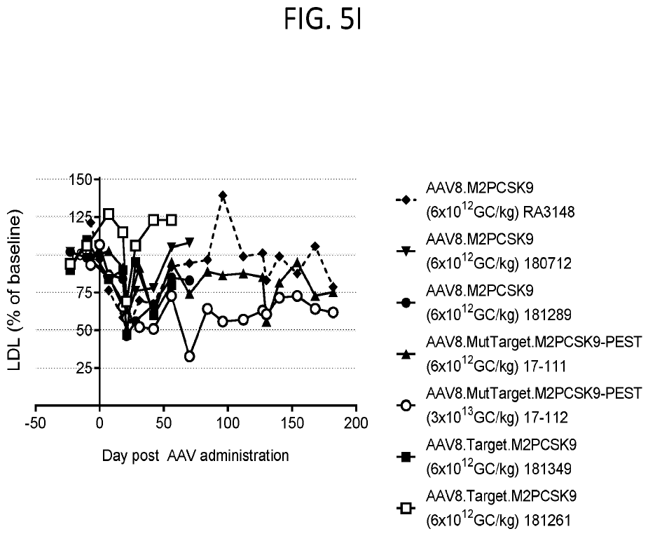

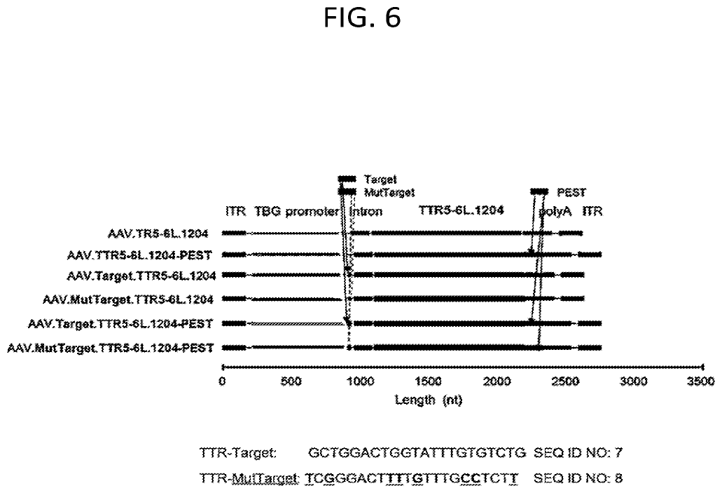

Despite a similar on-target editing (FIG. 6), the number of off-target sites identified by ITR-Seq was different among the groups. For the AAV.M2PCSk9 group the range was between 41 and 263 off-target sites (average 132). At the same dose, the off-targets for AAV.MutTarget.M2PCSK9+PEST group were 34 and for AAV.Target.M2PCSK9 the range was between 34 and 62 off-targets (average 48).

Preliminary results obtained in mouse and NHP experiments indicate that the AAV suicide system reduces M2PCSK9 off-target activity (FIG. 5E) while retaining on-target activity.

### Example 2—TTR

Using the techniques described in Example 1, with a TTR meganuclease or a TTR meganuclease — PEST fusion protein which recognizes the following site: SEQ ID NO: 7 -GCTGGACTGGTATTTGTGTCTG. See, FIG. 6.

### Example 3—ITR-Seq: a Next-Generation Sequencing Assay, Identifies Genome-Wide DNA Editing Sites In Vivo Following Genome Editing

The publication Breton et al, ITR-Seq, a next-generation sequencing assay, identifies genome-wide DNA editing sites in vivo following adeno-associated viral vector-mediated genome editing, BMC Genomics, (2020):21:239 is incorporated herein by reference in its entirety.

### Material and Methods

### Animal Studies

All animal procedures were performed in accordance with protocols approved by the Institutional Animal Care and Use Committee of the University of Pennsylvania.

### Rhesus Macaque Studies

DNA samples from previously published studies¹⁵ were used for ITR-Seq analysis. Briefly, AAV8 vectors driving the expression of the meganucleases M1PCSK9 (AAV8.TBG.M1PCSK9.WPRE) or M2PCSK9 (AAV8. TBG.M2PCSK9.WPRE) were administered via a peripheral vein to rhesus macaques (n=4 for M1PCSK9- and n=2 for M2PCSK9-treated animals). Liver biopsies were performed at 17 and 129 days (for AAV8-M 1PCSK9) or 18 and 128 days (for AAV8-M2PCSK9) post vector administration¹⁵. As an untreated control, we used DNA extracted from peripheral blood mononuclear cell (PBMC) samples collected prior to vector administration¹⁵.

To measure nuclease-independent AAV integration events, we analyzed liver DNA samples from a previous published study¹⁷. Briefly, one week or one month old male rhesus macaques were administered with AAV8.TBG.EGFP at a dose of 3×10¹² genome copies (GC)/kg. Animals were euthanized post-vector administration, and livers were collected.

### Mouse Studies

Newborn (0-2 days of age, n=2 per group) C57BL/6J mice were co-administered by temporal vein injection AAV expressing either SaCas9 (AAV8.TBG.hSaCas9.bGH), LbCpfl (AAV8.ABP2.TBG-S 1.hLbCpfl.bGH), or AsCpfl (AAV8.ABPS2.TBG-S1.hAsCpfl.PA75) at a dose of 3×10¹¹ GC/mouse, and vectors expressing specific sgRNA (AAV8.U6.sgRNA.mASS1.donor(mASS1)) or untargeted sgRNA as controls (AAV8.U6.sgRNA-ctrl.mASS1.donor(mASS1)) at a dose of 2×10¹² GC/mouse. At 21 days post-vector administration, mice were euthanized and livers collected.

Additional newborn mice (n=2 per group) were co-administered with vector expressing SaCas9 or LbCpfl as described above at a dose of 10¹¹ or 3×10¹¹ GC/mouse, with the second vector expressing ASS1-specific-sgRNA and the human coagulation factor IX (hFIX) transgene (AAV8.U6.sgRNA.mASS1.TBG.hFIX) at a dose of 10¹² GC/mouse. Livers were collected at 70 days post-vector administration.

### ITR-Seq

The developed ITR-Seq protocol is a modified version of an anchored PCR reaction^{18, 19}, in which a single primer is designed to anneal to and amplify outward from the ITR sequence (FIG. 7C). Following ITR integration in the DNA, the primer may be used to amplify the junction of the host genome and the inserted vector ITR sequence (FIGS. 7B, 7C). In order to adequately denature the ordered secondary structure of the integrated ITR, a high annealing temperature of 69° C. and longer adapter-specific primers were designed.

Amplicons were generated from purified genomic DNA isolated from liver tissue samples. DNA was sheared to an average size of 500 bp using an ME220 focused-ultrasonicator (Covaris, Woburn, Mass.), purified using AMPure beads (Beckman Coulter, Indianapolis, Ind.) at a 0.8× ratio, and eluted in 15 μl of elution buffer (Qiagen, Hilden, Germany). End repair was subsequently performed in a total volume of 22.5 μl containing 1 μl of 5 mM dNTP mix (Thermo Fisher Scientific, Waltham, Mass.), 2.5 μl of 10× SLOW ligation buffer (Enzymatics, Beverly, Mass.), 2 μl of End-Repair Mix (Low Concentration; Enzymatics, Beverly, Mass.), 2 μl of 10× buffer for Taq Polymerase (MgCl₂-free; Invitrogen, Carlsbad, Calif.), 0.5 μl of non-hot start Taq polymerase (New England BioLabs, Ipswich, Mass.), 0.5 μl of nuclease-free water (Life Technologies, Waltham, Mass.), and 14 μl of 400 ng sheared genomic DNA. The mix was incubated at 12° C. for 15 min, 37° C. for 15 min, 72° C. for 15 min, and held at 4° C. Unique Y-adapters, with molecular index tags annealed to MiSeq Common Adapters (Illumina, San Diego, Calif.), were ligated to the end-repaired DNA in the following mix: 1 μl of 10 μM annealed A01-A16 Y-adapter, 2 μl of T4 DNA ligase (Enzymatics, Beverly, Mass.), and 22.5 μl of the previous end-repaired DNA. The ligation program was 16° C. for 30 min, 22° C. for 30 min; reactions were held at 4° C. DNA was then purified by AMPure beads (Beckman Coulter, Indianapolis, Ind.) at a 0.7× ratio. End-repaired Y-adapter-ligated DNA fragments were amplified by PCR using an ITR-specific primer and an adapter-specific primer (A01-A16_P5_FWD primer) in the following mix (amounts per sample): 11.9 μl of nuclease-free water, 3 μl of 10× buffer for Taq Polymerase (MgCl₂-free, Invitrogen, Carlsbad, Calif.), 0.6 μl of 10 mM dNTP mix (Thermo Fisher Scientific, Waltham, Mass.), 1.2 μl of 50 mM MgCl₂ (Invitrogen, Carlsbad, Calif.), 0.3 μl of 5 U/μl Platinum Taq polymerase (Invitrogen, Carlsbad, Calif.), 1 μl of 10 μM GSP_ITR3.AAV2 primer, 1.5 μl of 0.5 M TMAC (Sigma-Aldrich, St. Louis, Mo.), 0.5 μl of 10 μM A01-A16_P5_FWD primer with the primer number matching the adapter number (e.g., A01_P5_FWD primer to be used with A01 Y-adapter), and 10 μl of previously purified DNA. The PCR program was 1 cycle of 95° C. for 5 min 30 cycles of 95° C. for 30 s, 69° C. for 1 min, and 72° C. for 30 s; 1 cycle at 72° C. for 5 min; 4° C. hold. PCR products were purified using 0.7× AMPure beads (Beckman Coulter, Indianapolis, Ind.) and resuspended in 15 μl of elution buffer (Qiagen, Hilden, Germany).

NGS libraries were prepared by PCR in the following mix (amounts per sample): 5.4 μl of nuclease-free water (Life Technologies, Waltham, Mass.), 3 μl of 10× buffer for Taq Polymerase (MgCl₂-free; Invitrogen, Carlsbad, Calif.), 0.6 μl of 10 mM dNTP mix (Thermo Fisher Scientific, Waltham, Mass.), 1.2 μl of 50 mM MgCl₂ (Invitrogen, Carlsbad, Calif.), 0.3 μl of 5 U/μl Platinum Taq polymerase (Invitrogen, Carlsbad, Calif.), 1 μl of 10 μM GSP_ITR3 primer, 1.5 μl of 0.5 M TMAC (Sigma-Aldrich, St. Louis, Mo.), 0.5 μl of 10 μM A01-A16_P5_FWD primer with the primer number matching the adapter number, 1.5 μl of 10 μM p701-16 primers, and 15 μl of previously purified DNA (including the AMPure beads used in the previous PCR purification step) The PCR program was 1 cycle of 95° C. for 5 min; 10 cycles of 95° C. for 30 sec, 75° C. for 2 min (−1° C./cycle), and 72° C. for 30 s; 15 cycles of 95° C. for 30 s, 69° C. for 1 min, and 72° C. for 30 s; 1 cycle at 72° C. for 5 min; 4° C. hold. PCR products were purified using 0.7× AMPure beads (Beckman Coulter, Indianapolis, Ind.), and resuspended in 25 μl of elution buffer. Dual-indexed sequencing libraries were sequenced on an Illumina MiSeq cartridge (MiSeq® v2 RGT Kit 300 cyc PE-Bx 1 of 2; San Diego, Calif.), generating 2×150 bp paired-end reads.

Sample demultiplexing and unique molecular identifier (UMI) tagging was performed on raw fastq files using Je²⁰, allowing for up to one mismatch on either index. Read pairs in which read 2 begins with the designed primer sequence, plus an additional flanking 20 bp of the AAV2 ITR sequence (FIG. 7A-7C) were identified using FASTX barcode slitter (http://hannonlab.cshl.edu/fastx_toolkit/, allowing for up to five mismatches), fastq-pair (https://github.com/linsalrob/EdwardsLab/), and FASTP²¹. Selected read pairs were mapped to the reference genome for each sample (MM10 for mouse and RheMac8 for rhesus macaque samples) using NovoAlign (Novocraft, Selangor, Malaysia). UMI read consolidation was subsequently performed using Je²⁰, resulting in a UMI consolidated BAM file. Chimeric reads spanning the ITR-genomic DNA insertion site were identified by determining split-read junctions for each read using SE-MEI (https://github.com/dpryan79/SE-MEI). Soft-clipped portions of reads were then mapped to the AAV2 reference genome using NovoAlign (Novocraft, Selangor, Malaysia). Only those originally mapped reads that were found to contain a soft-clipped read portion mapping to the AAV2 ITR with a mapping quality value greater than or equal to 30 were used to identify ITR integration sites by merging ITR integration sites found within a 50 bp window into a single ITR integration site using BEDtools^{22, 23}. Only those sites containing an ITR integration in both the forward and negative strand orientations were considered to be an identified off-target site. The genomic DNA sequence underlying each identified ITR integration site was aligned pairwise with the on-target DNA sequence motif from both the negative and positive strand orientations using the EMBOSS program' for semi-global alignment; this allowed assessment of sequence homology and the precise ITR integration site recognized by the nuclease of interest.

### Results and Discussion

### Developing the ITR-Seq Assay to Evaluate Meganuclease Activity in Non-Human Primates

The development of an NGS-based assay that can identify and rank nuclease-induced DSBs after in vivo gene editing significantly advances our ability to evaluate the safety and efficacy of genome editing therapies for translation to human clinical trials. Researchers have developed a variety of approaches to identify and quantify the on- and off-target activity of genome editing nucleases to better understand the elements that govern the nucleases' specificity and to improve the safety profile of these therapies²⁵. One can study the nuclease specificities and activities by first identifying the DSB, either in cultured cells or in animal models created as a consequence of their nuclease activity^{26,27.} Some of the approaches for determining this nuclease specificity include cell-free methods such as Site-Seq²⁸, Digenome-seq²⁹, and Circle-Seq³⁰. Some of the in vitro-based methods include GUIDE-See and Integrative-Deficient Lentiviral Vectors Capture (IDLV)^{31, 32}. These in vitro analyses, however, might not accurately predict the number and rate of off-target activity in vivo, since the conditions used for in vitro analysis are not representative of DNA accessibility and nuclease concentration present in the target organs of the animal models.

We therefore sought to develop a methodology for an unbiased, genome-wide identification of sites of ITR integration. By using the AAV ITR as a tag for identifying DSB, we can measure the off-target activity of genome-editing nucleases in vivo. Our method was based upon previous research that demonstrated AAV ITR sequence integration into the host's genomic DNA after DSBs have occurred^{10-14, 16, 33-36}.

Recently, our group characterized the genome-editing efficiency of a meganuclease that targeted the PCSK9 gene delivered to the liver of rhesus macaques using AAV8 vectors. This study showed stable, dose-dependent reductions of PCSK9 and an on-target indel percentage of 30-40% for the high and mid-level doses of the first-generation meganuclease, M1PCSK9, or the mid-dose of the second-generation meganuclease, M2PCSK9¹⁵. We isolated the edited PCSK9 alleles from the liver biopsy samples of rhesus macaques previously infused with both the first- and second-generation meganuclease constructs (AAV8-M1PCSK9 and AAV8-M2PCSK9, respectively)¹⁵. While analyzing these alleles using AMP-Seq and amplicon sequencing assays, we noted that the genomic integration of ITR sequences occurred at a high frequencyl¹⁵.

Here, we re-analyzed the NGS reads that were generated while characterizing the AAV8-M1PCSK9 and AAV8-M2PCSK9 on-target region¹⁵. Our aim was to identify the most common AAV ITR sequences that were integrated into the meganuclease on-target loci in the PCSK9 gene. Based on a peak in absolute frequency at position 82 of the AAV2 reference genome (FIG. 7A), we determined that the most frequent base position of ITR integration occurs 5′ upstream of the rep-binding element (RBE). We used this information to design an ITR-specific primer that hybridizes 5′ upstream of the observed ITR-integration start site (shown in red in FIG. 7B). This primer is used in a novel NGS assay, based upon a modified version of anchored multiplexed PCR, to identify the ITR-genomic DNA junction following insertional mutagenesis (FIG. 7C). We have called this method ITR-Seq.

To use the ITR-Seq assay for sample analysis, we first isolated DNA from the tissues of animals treated with nuclease-expressing AAV vectors. We sheared the DNA and ligated it to Y-adapters, as described in previous reports'. Following two rounds of PCR using the ITR-specific primer described above and adapter-specific primers, we produced NGS-compatible libraries. After sequencing, we computationally identified the resulting amplicons that contain both the amplified ITR sequence and adjacent genomic DNA sequences. We also determined the location and frequency of genome-wide ITR integration sites. By requiring that the ITR integrates in both the forward and reverse strand orientations at any particular identified-ITR integration site, we aimed to further reduce the number of false positive results and identify high-confidence ITR-integration sites. For each sample, we produced a rank-ordered list of nuclease target sites (ITR-Seq rank) and sorted the sites (in decreasing order) by the total number of observed ITR-integration events per locus (ITR-Seq reads).

### ITR-Seq Identifies Nuclease Off-Target Sites in Rhesus Macaques

Following the development of the ITR-Seq assay, we used this technique to further analyze the on- and off-target effects of meganucleases in rhesus macaques that had previously been administered AAV8-M1PCSK9 and AAV8-M2PCSK9 (FIGS. 8A-8C). As previously described, rhesus macaques received either one of three doses of AAV8-M1PCSK9 (3×10¹³ GC/kg, 6×10¹² GC/kg, or 2×10¹² GC/kg) or a single dose of AAV8-M2PCSK9 (6×10¹² GC/kg)¹⁵. We took a biopsy of the liver of all macaques on days 17 and 128 post-vector administration to evaluate on- and off-target editing. ITR-Seq reports, which were generated at the end of the computational analysis of the NGS data, include the most probable off-target sequence (based on the homology to the intended target sequence); the genomic location; and the ITR-Seq rank (according to the number of NGS reads mapping to the corresponding locus). Across all samples from meganuclease-treated macaques, the top ITR-Seq ranked site was the on-target locus (PCSK9 target site sequence TGGACCTCTTTGCCCCAGGGGA, chr1:54708864-54708885)¹⁵. For both generations of the meganuclease (i.e., AAV8-M1PCSK9 and AAV8-M2PCSK9), the number off-target sites identified by ITR-Seq depended on both the administered vector dose and the sample time point. The number of off-target sites decreased in a dose-dependent manner and as a function of time (e.g., day 17 had more off-target sites than day 128; see FIG. 8A). Animals that received the second-generation engineered meganuclease, M2PCSK9, had fewer identified off-target sites than animals treated with the first generation of the meganuclease, M1PCSK9, at the same dose. In total, we observed 1,170 different off-targets after administering AAV8-M1PCSK9 at a dose of 6×10¹² GC/kg. By contrast we only observed 194 and 105 off-targets in the two macaques that received AAV8-M2PCSK9 at a dose of 6×10¹² GC/kg (FIG. 8A).

A subset of ITR-Seq identified off-targets were randomly chosen from the results of d18 liver samples from macaques treated with AAV8-M1PCSK9 at a dose of 3×10¹³ and 6×10¹² GC/kg. The presence of ITR sequences was then investigated in these selected loci by AMP-Seq, as previously described^{15, 18}, using gene-specific primers flanking the identified off-target sequences. AMP-Seq results obtained by analysing liver DNA samples from d18 (from macaques treated with AAV8-M1PCSK9 at the same doses as specified above) are obtained (data not shown). For the analysed DNA, reads containing ITR sequences were found in 24 (for the 3×10¹³ GC/kg dose) or in 21 (for the 6×10¹² GC/kg dose) out of 27 interrogated loci, with the highest percentage of ITR integration (ITR-containing reads) corresponding to those off-targets with high ITR-Seq rank. Importantly, there is also an apparent correlation between the ITR-Seq rank, the percentage of ITR integration and the indel %, as in both animals the highest level of editing was shown in those loci with the highest ITR-Seq rank. For some of these loci we could not detect ITR sequences integrated by AMP-Seq assay, this could be due to a lower sensitivity for the AMP-Seq assay to detect ITR integration as the ITR-Seq method uses the actual ITR sequences as a starting point for the amplification. Similarly, we were able to observe ITR sequences in a couple of sites not shown in the ITR-Seq results (e.g. 20:359062-359285 and 7:165269225-165269449 for liver samples treated with AAV8-M1PCSK9 at a dose of 3×10¹³ GC/kg). These sites, however, were found in the ITR-Seq results in animals treated with AAV8-M1PCSK9 at a dose of 6×10¹² GC/kg, suggesting that our current protocol does not capture 100% of the ITR integration sites, and therefore the sensitivity of the ITR-Seq method can be improved.

We then annotated the identified sites of ITR integration based on the function of the DNA region (FIG. 7B). Regardless of the number of identified nuclease target sites, the genomic distribution of target sites (intergenic, intronic, or exonic regions) was reproducible amongst meganuclease-administered macaques. Generally, most target sites reside within introns, followed by intergenic regions of the genome (FIG. 8B). The meganucleases we evaluated here have a 22 nucleotide target site within the PCSK9 gene. Therefore, we evaluated the number of conserved nucleotides between the target DNA sequence and the DNA sequence of each off-target site (FIG. 8C). The distribution of matches to the on-target sequence appeared to follow a Gaussian distribution with a mean of 15-16 nucleotides. This indicates that the majority of ITR-Seq-identified off-target sites have between 6-7 mismatches between the targeted DNA sequence motif and the genomic DNA sequence of each target site (FIG. 8C). To assess if this level of homology resulted from editing happening in sequences similar to the meganuclease target sequence or simply due to chance, we generated 10 million random DNA regions (40 bp in length) within the rhesus macaque genome. We then attempted to identify the sequence that was most similar to the meganuclease target sites using the same algorithm used in the ITR-Seq protocol (dotted line, FIG. 8C). Unlike ITR-Seq-identified sequences, random sequences share an average of 11-12 mismatches with the intended target sequence. This indicates that ITR is mostly integrated in target with a certain degree of homology to the intended target sequence.

### Comparing GUIDE-Seq and ITR-Seq Identification of Off-Target Sites

The tools that identify nuclease on- and off-targets in vitro rely on the integration of an exogenous DNA in sites where DSB occurred. This exogenous DNA can be a double-stranded oligodeoxynucleotide (dsODN; as for GUIDE-Seq¹⁹) or a lentivirus genome (for Integrative-Deficient Lentiviral Vectors Capture, or IDLV^{31, 32}). Amplicon libraries can be constructed by PCR or LAM-PCR (linear-amplification mediated PCR) using adapter ligation and primers specific for these exogenous sequences. The location of this DSB can be identified at a later time by sequencing the constructed libraries using NGS followed by a bioinformatics analysis and mapping to reference genomes. One of the currently preferred methods for characterizing the off-target activity of nucleases is GUIDE-Seq because 1) it requires a minimal number of components; 2) this software is readily available to identify the off-targets; and 3) it can detect low abundance off-targets. As mentioned before, the nuclease activity in vitro might not be predictive of in vivo nuclease activity, considering that the dose, length of experiment, and cell type used in GUIDE-Seq are different from the specifics of animal models. GUIDE-Seq allows us to quickly compare multiple sgRNAs or guide RNA-independent nucleases, such as meganucleases. However, while it is possible to validate predicted off-targets using amplicon sequencing, one cannot identify novel, in vivo generated off-targets if they were not identified in vitro. Moreover, these methods require an in vivo validation step, where PCR amplicons, generated by primers encompassing the in vitro predicted off-targets regions, are later sequenced by NGS to calculate the rate of editing by indel identification.

For each meganuclease (M1PCSK9 and M2PCSK9), we had previously performed GUIDE-Seq analyses on LLC-MK2 cells transfected with plasmids to express the nucleases in order to identify off-target sites in vitro¹⁵. We selected the M1PCSK9 and M2PCSK9 off-target locations from the list of GUIDE-Seq-identified off-targets in vitro. Using rhesus macaques, we then performed an in vivo validation of these predicted off-targets using amplicon sequencing of off-target loci¹⁵. Both amplicon sequencing¹⁵ and ITR-Seq (FIG. 8A) exhibited a dose- and time-dependent reduction in off-target editing efficiency. We compared these previous results with our evaluation of off-target sites in vivo using ITR-Seq (FIGS. 9A-D). By identifying off-target sites with no homology to the indented target sequence, we were able to find approximately the same number of off-target sites across two, independent experiments using either M1PCSK9 (1093 and 1499, for GUIDE-Seq experiment 1 and 2, respectively) or M2PCSK9 (568 and 651, for GUIDE-Seq experiment 1 and 2, respectively). We compared sites that were identified by both Guide-Seq in vitro experiments to the off-target sites identified by ITR-Seq. For this study in rhesus macaques, we performed ITR-Seq on DNA samples from liver biopsies taken on day 17 post-nuclease administration. We compared the GUIDE-Seq and ITR-Seq-identified off-targets in macaques that received a dose of 3×10¹³ GC/kg of AAV8-M1PCSK9 (FIG. 9A), 6×10¹² GC/kg of AAV8-M1PCSK9 (FIG. 9B), and two animals administered with 6×10¹² GC/kg of AAV8-M2PCSK9 (FIGS. 9C and 9D). Most (71.9-82.9%) off-target sites identified exclusively by ITR-Seq, and not by Guide-Seq (see colored sections of FIGS. 9A-D). Interestingly, among animals that received the second-generation nuclease (M2PCSK9) both ITR-Seq and Guide-Seq identified fewer off-target sites (see white sections of FIGS. 9A-D).

We have previously validated a set of GUIDE-Seq-identified high- and low-rank off-targets, which were ranked according to the number of reads¹⁵. We did this by quantifying the indel percentage, which we determined using amplicon sequencing of off-target loci in samples taken from macaques administered with meganucleases on days 17/18 and 128/129¹⁵. Off-target sites that exhibited a significantly higher indel percentage than untreated PBMC DNA control samples were counted as positive.

Here, we evaluated whether ITR-Seq can identify the positive GUIDE-Seq off-targets. Given that we re-analysed the same DNA used to validate the GUIDE-Seq off-targets, we were able to assess the sensitivity of our method in detecting the positive off-target loci. Among macaques administered with 3×10¹³ GC/kg and 6×10¹² GC/kg of AAV8-M1PCSK9, ITR-Seq failed to identify only two high-rank and two low-rank positive GUIDE-Seq off-targets. Among macaques administered with AAV8-M2PCSK9, ITR-Seq correctly identified most of the positive off-targets and only missed three low-rank positive off-targets in one animal and three high-rank positive off-targets in the other macaque. Taken together, these results clearly indicate that the vast majority of in vivo off-targets were only identified by ITR-Seq and not by GUIDE-Seq. This suggests that unlike amplicon sequencing of GUIDE-Seq-predicted off-targets, ITR-Seq provides a more accurate examination of the activity of AAV-delivered nucleases in vivo.

ITR-Seq analysis on DNA samples from macaques administered with AAV8-M1PCSK9 and AAV8-M2PCSK9 revealed that it is possible to characterize the off-target sites of a guide RNA-independent nuclease in vivo. ITR-Seq has the potential to provide a detailed characterization of the genome-editing nucleases. Furthermore, in vitro off-target data is not comprehensive of the genome-editing nuclease off-target activity in vivo. Indeed, ITR-Seq not only identified most of the GUIDE-Seq-identified top rank off-target sites we characterized in our previous study¹⁵, but also identified other off-targets that were not captured by GUIDE-Seq assays in vitro (FIGS. 9A-9D). By using the AAV ITR as a tag for identifying DSB, we believe that the ITR-Seq method captures true off-targets as several sites were previously identified by Guide-Seq and amplicon sequencing. Moreover, the sequences identified by ITR-Seq are similar to the intended target sequence. Therefore, ITR-Seq can identify novel off-targets that were previously undetected by the combined approach of Guide-Seq and subsequent amplicon sequencing.

In contrast to Guide-Seq, ITR-Seq is not a tool for predicting off-targets. Rather, ITR-Seq identifies novel sites in the genome where on- and off-target nuclease activity occurred. Indeed, this method identifies AAV ITR integrations sites directly from the DNA samples of animals treated with nuclease-expressing AAV. Identified off-targets can be further analysed using amplicon sequencing to 1) accurately determine the percent of editing and ITR integration; and 2) obtain a detailed panorama of the nuclease activity in clinically relevant doses and animal models.

### Evaluating ITR-Seq Assay as a Tool for Identifying Guide RNA-Dependent Nuclease Off-Target Sites in Mice

We tested whether ITR-Seq can detect the on- and off-target activity of a variety of guide RNA-dependent nucleases (i.e., SaCas9, LbCpfl, and AsCpfl) that are commonly used in preclinical studies. We also wanted to evaluate whether ITR-Seq analysis is compatible with the distinct types of DSB ends created by these nucleases (blunt for SaCas9 and 5′ overhang for Cpfl).

Newborn C57BL6/J mice were co-administered vectors expressing SaCas9, LbCpfl, or AsCpfl nucleases at a dose of 3×10¹¹ GC/mouse together with vectors expressing the corresponding guide RNAs at a dose of 2×10¹² GC/mouse (sgRNA, Table 2). Mice were euthanized on day 21 post-vector administration. The liver was harvested and the DNA was extracted for ITR-Seq analysis in order to evaluate the frequency and location of nuclease-mediated DNA cleavage sites. Additional groups of newborn mice were co-administered vectors expressing SaCas9 or LbCpfl as above at a dose of 10¹¹ or 3×10¹¹ GC/mouse. The second vector expressed sgRNA and the hFIX transgene (instead of the donor DNA sequence used in the first experiment) at a dose of 1×10² GC/mouse. Our goal was to evaluate the effect of vector dose on ITR integration. These mice were euthanized on day 70 post-vector administration. DNA samples from the liver were then subjected to ITR-Seq analysis (Table below).

Aside from one animal treated with AsCpfl-sgRNA2, the on-target locus (mASS1) was the target with the highest number of reads in ITR-Seq analysis. In addition, all of the evaluated sgRNA-directed nucleases exhibited high specificity for the targeted locus with an on-target indel percentage of up to 33% (Table 2). Importantly, mice treated with AAV8-SaCas9 exhibited the highest frequency of on-target ITR integration events across all treated samples (Table 2 below). Despite showing comparable on-target editing, we observed lower on-target ITR integration events in livers treated with AAV8-LbCpfl versus AAV8-SaCas9. AAV8-AsCpfl had very low editing efficiency at the on-target locus with a maximum indel percentage of 1.22%, as assessed by targeted amplicon sequencing (Table 2 below).

| Vector 1 | Vector 2 | Time | Indel % | | Off-Target | | | |
|---|---|---|---|---|---|---|---|---|
| (GC/kg | (GC/kg | point | (on-target) | | Sites | | | |
| dose) | dose) | (days) | Mouse A | Mouse B | Mouse A | Mouse B | Target locus | Target Sequence |
| AAV8-SaCas9 | AAV8-sgRNA1 | 21 | 28.05 | 32.59 | 3 | 4 | ASS1 | ACAGGACTCCCAGAG |
| (3 × 10¹¹) | (2 × 10¹²) | | | | | | (c5r2:31518639-31518658) | TTAGA |
| AAV8-LbCpf1 | AAV8-sgRNA1 | 21 | 14.35 | 23.87 | 1 | 2 | ASS1 | CAAATGGCAGGAAGA |
| (3 × 10¹¹) | (2 × 10¹²) | | | | | | (chr2:31518480-31518502) | ATTCACGG |
| AAV8-LbCpf1 | AAV8-sgRNA2 | 21 | 33.42 | 27.64 | 4 | 3 | ASS1 | TGGCTGGAAATATTA |
| (3 × 10¹¹) | (2 × 10¹²) | | | | | | (chr2:31519012-31519034) | GGGCAACT |
| AAV8-AsCpf1 | AAV8-sgRNA1 | 21 | 1.22 | 0.38 | 3 | 1 | ASS1 | CAAATGGCAGGAAGA |
| (3 × 10¹¹) | (2 × 10¹²) | | | | | | (chr2:31519480-31518502) | ATTCACGG |
| AAV8-AsCpf1 | AAV8-sgRNA2 | 21 | 0.28 | 0.12 | 2 | 1 | ASS1 | TGGCTGGAAATATTA |
| (3 × 10¹¹) | (2 × 10¹²) | | | | | | (c5r2:31519012-31519034) | GGGCAACT |
| AAV8-SaCas9 | AAV8-sgRNA1 | 70 | 26.94 | 29.11 | 10 | 12 | ASS1 | ACAGGACTCCCAGAG |
| (3 × 10¹¹) | (1 × 10¹²) | | | | | | (chr2:31518639-31518658) | TTAGA |
| AAV8-SaCas9 | AAV8-sgRNA1 | 70 | 21.39 | 25.98 | 7 | 9 | ASS1 | ACAGGACTCCCAGAG |
| (1 × 10¹¹) | (1 × 10¹²) | | | | | | (chr2:31518639-31518658) | TTAGA |
| AAV8-LbCpf1 | AAV8-sgRNA2 | 70 | 4.91 | 4.90 | 5 | 6 | ASS1 | TGGCTGGAAATATTA |
| (1 × 10¹¹) | (1 × 10¹²) | | | | | | (chr2:31519012-31519034) | GGGCAACT |
| AAV8-LbCpf1 | AAV8-sgRNA2 | 70 | 7.69 | 8.48 | 10 | 4 | ASS1 | TGGCTGGAAATATTA |
| (3 × 10¹¹) | (1 × 10¹²) | | | | | | (chr2:31519012-31519034) | GGGCAACT |

All of the ITR-Seq-identified off-target sites for the CRISPR nucleases resided within annotated mouse genes, with the most common site being the on-target locus (data not shown). We identified some low-frequency off-targets for the tested sgRNAs, most of which had low homology to the target sequence. An off-target site identified for the AAV8-SaCas9 sgRNA was located within the locus of a known oncogene, NOTCH2 (data not shown). Importantly, this off-target was found in mice administered with AAV8-SaCas9 at a dose of 3×10¹¹ GC/mouse and AAV-sgRNA1 at a dose of 2×10¹² GC/mouse. However, this off-target was absent following administration of 10¹² GC/mouse of AAV-sgRNA, a two-fold lower dose. We needed a higher dose of AAV8-sgRNA1 to observe editing at this low abundance off-target (Table 2). The rate of AAV ITR integration can be influenced by 1) the homology between the target sequence; or 2) the blunt or overhang nature of the DNA ends as a result of the nuclease cuts. We need to conduct detailed studies in future to fully understand the dynamics of ITR integration.

### Analysing Nuclease-Independent Events in Mice and Non-Human Primates

Among mice injected with AAV8 expressing a CRISPR-related nuclease and a sgRNA, ITR integration occurred in the loci targeted by the sgRNA. Interestingly, ITR integration also occurred in a seemingly sgRNA-independent fashion as we observed AAV ITR integration in control samples where there was no functional sgRNA. In mice, the most common nuclease-independent ITR integration events occurred in the Gm10800 and albumin genes. ITR integration in the albumin gene concurs with previous reports, which show that the albumin gene is quite susceptible to AAV integration³⁷. AAV integration has been reported for genes that are transcriptionally active in the liver³⁸.

To evaluate the frequency and scope of nuclease-independent ITR-integration in non-human primates, we evaluated the liver DNA of rhesus macaques administered with AAV-eGFP at a dose of 3×10¹² GC/kg. We detected nuclease-independent integration events, although not in identified genes, as positive ITR insertions. Importantly, our method appears to exclusively identify integrated ITR sequences as a result of in vivo events. We have come to this conclusion based on the finding that analysing DNA isolated from the PBMCs of untreated rhesus macaques spiked with AAV.eGFP DNA did not result in the identification of off-targets (data not shown).

While the observed ITR integration is the result of nuclease-induced DSB, other researchers have shown that DSB induced by restriction enzymes, drugs, or gamma-irradiation can also result in AAV-ITR insertions at these break points¹⁶. Identifying and characterizing nuclease-independent AAV integration sites is especially important to create a more complete AAV safety profile, not only for genome editing, but also for gene-therapy studies.

Alternative methods such as BLISS⁶, BLESS^{39, 40}, and End-Seq⁷ can identify sites of DSB by capturing the DSB ends created by the activity of the nuclease. These methods can accurately identify nuclease-induced DSB in vivo. However, these methods have the limitation that they can only capture the DSB present at a single time point. This limitation can be partially overcome by analysing DSB at multiple time points. Non-restrictive LAM-PCR coupled with NGS⁴¹, a method similar to ITR-Seq, can detect AAV-ITR integration sites. In this technique, a linear amplification is first performed using an ITR-specific biotinylated primer. This single-stranded DNA is isolated by streptavidin beads and ligated to a known adapter. Using secondary LTR-specific and adapter-specific primers, one can amplify, sequence, and identify the region of LTR integration⁴¹. This method was used to identify the integration sites of AAV1-LPLS447X vector, which was developed for treating lipoprotein lipase deficiency (LPLD) in mouse and human DNA samples³⁴. Although in theory nrLAM-PCR can detect the off-target activity of nucleases, researchers have not yet undertaken a direct comparison of ITR-Seq and nrLAM-PCR to understand the advantages and limitations of these two techniques.

### Conclusion

In conclusion, we have developed a NGS assay to assess the specificity of any AAV-expressed nucleases in vivo. The ITR-Seq assay can be used to identify AAV insertion sites in vivo in gene-editing studies. If locus-specific analyses are needed, such as calculating indel percentage in the identified off-target region or characterizing the integrated ITR sequences, then deep-sequencing analysis is recommended. It is important, however, to keep in mind that detection of ITR integration events by ITR-Seq is more sensitive than other NGS-based methods such as AMP-Seq.

Given that the only requirement is using an AAV as a delivery vector, ITR-Seq can be used to measure the specificity of the nucleases in virtually any organism with an annotated reference genome. ITR-Seq may be used as a companion diagnostic in pre-clinical and clinical studies to evaluate nuclease target sites in longitudinal animal studies that have varying dosages and/or administration routes. This technique can yield invaluable insights into the safety and efficacy of gene-editing therapies and ultimately better inform the design of gene-editing therapies.

This same approach can evaluate AAV-integration events in traditional AAV gene-therapy studies. While the risks of insertional mutagenesis from AAV gene therapy is considered low^{8, 42} its use is justifiable for treating rare disabling and lethal diseases, these potential risks may be more relevant as the field evolves to treating less severe acquired diseases.

The following list of references correspond to the documents in Example 3.

### Example 4 — AAV.PCSK9 Self-Inactivating Vectors

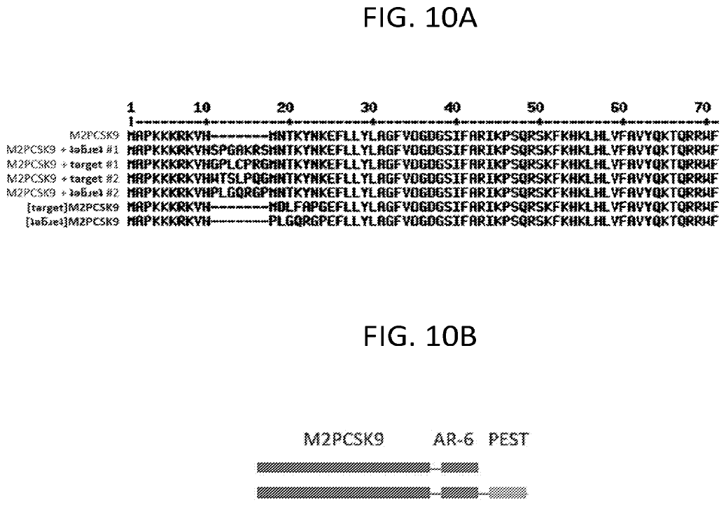

FIGS. 10A-10B show embodiments in which a target sequence or mutant target sequence is inserted in the enzyme coding sequence. FIG. 10A is an amino acid alignment showing a 10-amino acid nuclear localization signal (NLS) followed by the protein expressed from a target sequence (amino acids 10-20, followed by the active portion of the nuclease. The first amino acid sequence M2PCSK9 shows a fragment of a reference meganuclease with its NLS, a space noting where the other constructs will have insertions, and the sequence of the nuclease beginning at position 18. M2PCSK9[inverted target #1] shows the protein encoded when a target sequence is inserted on the anti-sense strand between the NLS and the enzyme. M2PCSK9[target #1] shows the protein encoded when a target sequence inserted on the sense strand between the NLS and the enzyme. M2PCSK9 [target #2] shows the protein encoded when a different target sequence is inserted on the sense strand between the NLS and the enzyme. M2PCSK9 [inverted target #2] shows the protein encoded when the (mut)target sequence is inserted on the anti-sense strand between the NLS and the enzyme. [target]M2PCSK9 shows the protein encoded when the 22 bp target sequence replaces the coding sequences after the NLS, resulting in substitution of the six amino acids of the enzyme. [invertedtarget]M2PCSK9 shows the protein encoded when the 22 bp target sequence replaces the coding sequences after the NLS on the opposite strand, resulting in replacement of the seven amino acids of the enzyme. FIG. 10B shows the design of a PCSK9 meganuclease fusion protein, one having a single protein degradation signal (degron) which is ubiquitin-independent (AR-6), and a PCSK9 meganuclease fusion protein having two protein degradation signals, AR-6 and PEST.

All documents cited in this specification are incorporated herein by reference, as are sequences and text of the Sequence Listing filed herewith are incorporated by reference. While the invention has been described with reference to particular embodiments, it will be appreciated that modifications can be made without departing from the spirit of the invention. Such modifications are intended to fall within the scope of the appended claims.

Sequence Listing Features:

| Sequence | <223> Feature | |
|---|---|---|
| 1 | <223> | CRE motif |
| 2 | <223> | CRE Recognition sequence |
| 3 | <223> | PEST sequence |
| 4 | <223> | Synthetic Construct |
| 5 | <223> | PCSK9 target |
| 6 | <223> | Mutant Target sequence |
| | | for PCSK9 meganuclease |
| 7 | <223> | TTR Target |
| 8 | <223> | TTR Mutant Target |
| 9 | <223> | PCSK7-8L.197 |
| 10 | <223> | Synthetic Construct |
| 11 | <223> | TBG.Target.PI.M2PCSK9 |
| | | Nuclease.bGH |
| | <221> | misc_feature |
| | <222> | (l) . . . (168) |
| | <223> | ITR |
| | <220> | |
| | <221> | misc_feature |
| | <222> | (211) . . . (907) |
| | <223> | Promoter/enhancer |
| | <220> | |
| | <221> | misc_feature |
| | <222> | (914) . . . (395) |
| | <223> | Target |
| | <220> | |
| | <221> | misc_feature |
| | <222> | (961) . . . (1093) |
| | <223> | Intron |
| | <220> | |
| | <221> | misc_feature |
| | <222> | (1110) . . . (2204) |
| | <223> | M2PCKS9 CDS |
| | <220> | |
| | <221> | misc_feature |
| | <222> | (2211) . . . (2425) |
| | <223> | bovine growth hormone (bGH) polyA |
| | <220> | |
| | <221> | misc_feature |
| | <222> | (2475) . . . (2642) |
| | <223> | ITR |
| 12 | <223> | TBG.Target.PI.PCS9 Nuclease-PEST |
| | <221> | misc_feature |
| | <222> | (l) . . . (168) |
| | <223> | ITR |
| | <220> | |
| | <221> | misc_feature |
| | <222> | (211) . . . (907) |
| | <223> | promoter/enhancer |
| | <220> | |
| | <221> | misc_feature |
| | <222> | (914) . . . (935) |
| | <223> | target |
| | <220> | |
| | <221> | misc_feature |
| | <222> | (961) . . . (1093) |
| | <223> | intron |
| | <220> | |
| | <221> | misc_feature |
| | <222> | (1109) . . . (2200) |
| | <223> | M2PCSK9 CDS |
| | <220> | |
| | <221> | misc_feature |
| | <222> | (2201) . . . (2332) |
| | <223> | PEST |
| | <220> | |
| | <221> | misc_feature |
| | <222> | (2333) . . . (2547) |
| | <223> | bovine growth hormone (bGH) polyA |
| | <220> | |
| | <221> | misc_feature |
| | <222> | (2597) . . . (2764) |
| | <223> | ITR |
| 13 | <223> | TBG.Target.PI.PCS9 |
| | | Nuclease-PEST.Target.bGH |
| | <221> | repeat_region |
| | <222> | (l) . . . (168) |
| | <223> | ITR |
| | <220> | |
| | <221> | promoter |
| | <222> | (211) . . . (907) |
| | <223> | TBG promoter |
| | <220> | |
| | <221> | enhancer |
| | <222> | (211) . . . (310) |
| | <223> | alpha/mic/bik enhancer |
| | <220> | |
| | <221> | enhancer |
| | <222> | (317) . . . (416) |
| | <223> | enhancer 2 |
| | <220> | |
| | <221> | misc_feature |
| | <222> | (914) . . . (935) |
| | <223> | Target sequence |
| | <220> | |
| | <221> | Intron |
| | <222> | (961) . . . (1093) |
| | <220> | |
| | <221> | misc_feature |
| | <222> | (1110) . . . (2201) |
| | <223> | PCSK7-8L.197 coding sequence |
| | <220> | |
| | <221> | misc_feature |
| | <222> | (2202) . . . (2330) |
| | <223> | PEST |
| | <220> | |
| | <221> | misc_feature |
| | <222> | (2334) . . . (2355) |
| | <223> | target |
| | <220> | |
| | <221> | polyA_signal |
| | <222> | (2361) . . . (2575) |
| | <223> | bovine growth hormone (bGH) polyA |
| | <220> | |
| | <221> | repeat_region |
| | <222> | (2625) . . . (2792) |
| | <223> | 3′ ITR |
| 14 | <223> | TBG.MutTarget.PI.Nuclease-PEST.bGH |
| | <221> | misc_feature |
| | <222> | (1) . . . (168) |
| | <223> | ITR |
| | <220> | |
| | <221> | misc_feature |
| | <222> | (211) . . . (907) |
| | <223> | Promoter/enhancer |
| | <220> | |
| | <221> | misc_feature |
| | <222> | (914) . . . (935) |
| | <223> | mutant target |
| | <220> | |
| | <221> | misc_feature |
| | <222> | (961) . . . (1093) |
| | <223> | intron |
| | <220> | |
| | <221> | misc_feature |
| | <222> | (1110) . . . (2201) |
| | <223> | M2PSCK9 CDS |
| | <220> | |
| | <221> | misc_feature |
| | <222> | (2202) . . . (2333) |
| | <223> | PEST CDS |
| | <220> | |
| | <221> | misc_feature |
| | <222> | (2334) . . . (2548) |
| | <223> | bGH Poly A |
| | <220> | |
| | <221> | misc_feature |
| | <222> | (2598) . . . (2765) |
| | <223> | ITR |
| 15 | <223> | TBG.PI.PCSK9 Nuclease-PEST |
| | <221> | misc_feature |
| | <222> | (1) . . . (168) |
| | <223> | ITR |
| | <220> | |
| | <221> | misc_feature |
| | <222> | (211) . . . (907) |
| | <223> | Promoter/enhancer |
| | <220> | |
| | <221> | misc_feature |
| | <222> | (939) . . . (1071) |
| | <223> | intron |
| | <220> | |
| | <221> | misc_feature |
| | <222> | (1089) . . . (2180) |
| | <223> | M2PCSK9 CDS |
| | <220> | |
| | <221> | misc_feature |
| | <222> | (2181) . . . (2312) |
| | <223> | PEST CDS |
| | <220> | |
| | <221> | misc_feature |
| | <222> | (2313) . . . (2527) |
| | <223> | bGH Poly A |
| | <220> | |
| | <221> | misc_feature |
| | <222> | (2577) . . . (2744) |
| | <223> | ITR |
| 16 | <223> | Vector genome: TBG.MutTarget. |
| | | PI.PCS9 Nuclease.bGH |
| | <221> | misc_feature |
| | <222> | (l) . . . (168) |
| | <223> | ITR |
| | <220> | |
| | <221> | misc_feature |
| | <222> | (211) . . . (907) |
| | <223> | promoter/enhancer |
| | <220> | |
| | <221> | misc_feature |
| | <222> | (914) . . . (935) |
| | <223> | mutant target |
| | <220> | |
| | <221> | misc_feature |
| | <222> | (961) . . . (1093) |
| | <223> | intron |
| | <220> | |
| | <221> | misc_feature |
| | <222> | (1110) . . . (2204) |
| | <223> | M2PCSK9 CDS |
| | <220> | |
| | <221> | misc_feature |
| | <222> | (2215) . . . (2429) |
| | <223> | bGH Poly A |
| | <220> | |
| | <221> | misc_feature |
| | <222> | (2479) . . . (2646) |
| | <223> | ITR |
| 17 | <223> | PCSK9 Meganuclease - PEST fusion |
| 18 | <223> | Synthetic Construct |
| 19 | <223> | Primer |
| 20 | <223> | Primer |
| 21 | <223> | Primer |
| 22 | <223> | Primer |
| 23 | <223> | Primer |
| 24 | <223> | Primer |
| 25 | <223> | Primer |
| 26 | <223> | Primer |

### REFERENCES

1. Kosicki, M., Tomberg, K. & Bradley, A. Repair of double-strand breaks induced by CRISPR-Cas9 leads to large deletions and complex rearrangements. Nat Biotechnol 36, 765-771 (2018).

2. Sander, J. D. & Joung, J. K. CRISPR-Cas systems for editing, regulating and targeting genomes. Nat Biotechnol 32, 347-355 (2014).

3. Waryah, C. B., Moses, C., Arooj, M. & Blancafort, P. Zinc Fingers, TALEs, and CRISPR Systems: A Comparison of Tools for Epigenome Editing. Methods Mol Biol 1767, 19-63 (2018).

4. Arnould, S. et al. The I-CreI meganuclease and its engineered derivatives: applications from cell modification to gene therapy. Protein Eng Des Sel 24, 27-31 (2011).

5. Silva, G. et al. Meganucleases and other tools for targeted genome engineering: perspectives and challenges for gene therapy. Curr Gene Ther 11, 11-27 (2011).

6. Yan, W. X. et al. BLISS is a versatile and quantitative method for genome-wide profiling of DNA double-strand breaks. Nat Commun 8, 15058 (2017).

7. Canela, A. et al. DNA Breaks and End Resection Measured Genome-wide by End Sequencing. Mol Cell 63, 898-911 (2016).

8. Chandler, R. J., Sands, M. S. & Venditti, C. P. Recombinant Adeno-Associated Viral Integration and Genotoxicity: Insights from Animal Models. Hum Gene Ther 28, 314-322 (2017).

9. Janovitz, T., Oliveira, T., Sadelain, M. & Falck-Pedersen, E. Highly divergent integration profile of adeno-associated virus serotype 5 revealed by high-throughput sequencing. J Vivol 88, 2481-2488 (2014).

10. Kotin, R. M. et al. Site-specific integration by adeno-associated virus. Proc Natl Acad Sci USA 87, 2211-2215 (1990).

11. Miller, D. G. et al. Large-scale analysis of adeno-associated virus vector integration sites in normal human cells. J Vivol 79, 11434-11442 (2005).

12. Nakai, H. et al. Large-scale molecular characterization of adeno-associated virus vector integration in mouse liver. J Vivol 79, 3606-3614 (2005).

13. Nault, J. C. et al. Recurrent AAV2-related insertional mutagenesis in human hepatocellular carcinomas. Nat Genet 47, 1187-1193 (2015).

14. Samulski, R. J. et al. Targeted integration of adeno-associated virus (AAV) into human chromosome 19. EMKO J10, 3941-3950 (1991).

15. Wang, L. et al. Meganuclease targeting of PCSK9 in macaque liver leads to stable reduction in serum cholesterol. Nat Biotechnol 36, 717-725 (2018).

16. Miller, D. G., Petek, L. M. & Russell, D. W. Adeno-associated virus vectors integrate at chromosome breakage sites. Nat Genet 36, 767-773 (2004).

17. Wang, L. et al. AAV8-mediated hepatic gene transfer in infant rhesus monkeys (Macaca mulatta). Mol Ther 19, 2012-2020 (2011).

18. Zheng, Z. et al. Anchored multiplex PCR for targeted next-generation sequencing. Nat Med 20, 1479-1484 (2014).

19. Tsai, S. Q. et al. GUIDE-seq enables genome-wide profiling of off-target cleavage by CRISPR-Cas nucleases. Nat Biotechnol 33, 187-197 (2015).

20. Girardot, C., Scholtalbers, J., Sauer, S., Su, S. Y. & Furlong, E. E. Je, a versatile suite to handle multiplexed NGS libraries with unique molecular identifiers. BMC Bioinformatics 17, 419 (2016).

21. Chen, S., Zhou, Y., Chen, Y. & Gu, J. fastp: an ultra-fast all-in-one FASTQ preprocessor. Bioinformatics 34, i884-i890 (2018).

22. Quinlan, A. R. BEDTools: The Swiss-Army Tool for Genome Feature Analysis. Current protocols in bioinformatics 47, 11.12.11-34 (2014).

23. Quinlan, A. R. BEDTools: The Swiss-Army Tool for Genome Feature Analysis. Curr Protoc Bioinformatics 47, 11 12 11-34 (2014).

24. Rice, P., Longden, I. & Bleasby, A. EMBOSS: the European Molecular Biology Open Software Suite. Trends Genet 16, 276-277 (2000).

25. Martin, F., Sanchez-Hernandez, S., Gutierrez-Guerrero, A., Pinedo-Gomez, J. & Benabdellah, K. Biased and Unbiased Methods for the Detection of Off-Target Cleavage by CRISPR/Cas9: An Overview. Int J Mol Sci 17 (2016).

26. Lazzarotto, C. R. et al. Defining CRISPR-Cas9 genome-wide nuclease activities with CIRCLE-seq. Nat Protoc 13, 2615-2642 (2018).

27. Yee, J. K. Off-target effects of engineered nucleases. FEBS J 283, 3239-3248 (2016).

28. Cameron, P. et al. Mapping the genomic landscape of CRISPR-Cas9 cleavage. Nat Methods 14, 600-606 (2017).

29. Kim, D. et al. Digenome-seq: genome-wide profiling of CRISPR-Cas9 off-target effects in human cells. Nat Methods 12, 237-243, 231 p following 243 (2015).

30. Tsai, S. Q. et al. CIRCLE-seq: a highly sensitive in vitro screen for genome-wide CRISPR-Cas9 nuclease off-targets. Nat Methods 14, 607-614 (2017).

31. Gabriel, R. et al. An unbiased genome-wide analysis of zinc-finger nuclease specificity. Nat Biotechnol 29, 816-823 (2011).

32. Wang, X. et al. Unbiased detection of off-target cleavage by CRISPR-Cas9 and TALENs using integrase-defective lentiviral vectors. Nat Biotechnol 33, 175-178 (2015).

33. Donsante, A. et al. AAV vector integration sites in mouse hepatocellular carcinoma. Science 317, 477 (2007).

34. Kaeppel, C. et al. A largely random AAV integration profile after LPLD gene therapy. Nat Med 19, 889-891 (2013).

35. Kotin, R. M., Menninger, J. C., Ward, D. C. & Berns, K. I. Mapping and direct visualization of a region-specific viral DNA integration site on chromosome 19q13-qter. Genomics 10, 831-834 (1991).

36. Rosas, L. E. et al. Patterns of scAAV vector insertion associated with oncogenic events in a mouse model for genotoxicity. Mol Ther 20, 2098-2110 (2012).

37. Chandler, R. J. et al. Vector design influences hepatic genotoxicity after adeno-associated virus gene therapy. J Clin Invest 125, 870-880 (2015).

38. Nakai, H. et al. AAV serotype 2 vectors preferentially integrate into active genes in mice. Nat Genet 34, 297-302 (2003).

39. Crosetto, N. et al. Nucleotide-resolution DNA double-strand break mapping by next-generation sequencing. Nat Methods 10, 361-365 (2013).

40. Ran, F. A. et al. In vivo genome editing using Staphylococcus aureus Cas9. Nature 520, 186-191 (2015).

41. Paruzynski, A. et al. Genome-wide high-throughput integrome analyses by nrLAM-PCR and next-generation sequencing. Nat Protoc 5, 1379-1395 (2010). 42. Mingozzi, F. & High, K. A. Therapeutic in vivo gene transfer for genetic disease using AAV: progress and challenges. Nat Rev Genet 12, 341-355 (2011).

What is claimed is:

1. A self-modulating gene editing nuclease expression cassette comprising:
(a) a nucleic acid sequence comprising a nuclease coding sequence which is operably linked to regulatory sequences which direct expression of the nuclease following delivery to a host cell having a sequence to which the nuclease is targeted;
(b) at least one nuclease modulating sequence which is selected from the target sequence for the nuclease or a mutated target sequence which is recognized by the nuclease following its expression.

2. The self-modulating nuclease expression cassette according to claim 1, wherein the nucleic acid sequence of (a) further comprises a coding sequence for peptide degradation signal which is in frame with the nuclease coding sequence such the nuclease-peptide degradation signal is expressed as a fusion protein.

3. The self-modulating nuclease expression cassette according to claim 1 or 2, wherein the expression cassette comprises a mutated target sequence.

4. The self-modulating nuclease expression cassette according to any one of claims 1 to 3, wherein a first nuclease modulating sequence is located upstream of the nuclease coding sequence.

5. The self-modulating nuclease expression cassette according to any one of claims 1 to 4, wherein a nuclease modulating sequence is located downstream of the nuclease coding sequence.

6. The self-modulating nuclease expression cassette according to any one of claims 1 to 4, wherein a nuclease modulating sequence is located within the nuclease coding sequence.

7. The self-modulating nuclease expression cassette according to any one of claims 1 to 6, wherein the expression cassette comprises at least two nuclease modulating sequences.

8. The self-modulating nuclease expression cassette according to any one of claims 1 to 6 or claim 7, wherein the expression cassette comprises at least two different nuclease modulating sequences.

9. The self-modulating nuclease expression cassette according to claim 8, wherein the at least two different nuclease modulating sequences have different mutated target sequences.

10. The self-modulating nuclease expression cassette according to any one of claims 1 to 9, wherein each nuclease modulating sequence is independently 10 to 40 nucleotides in length.

11. The self-modulating nuclease expression cassette according to any one of claims 1 to 9, wherein the nuclease is a meganuclease and each nuclease modulating sequence is about 22 nucleotides in length.

12. The self-modulating nuclease expression cassette according to any one of claims 1 to 11, wherein the protein degradation signal is 10 amino acids to 50 amino acids in length.

13. The self-modulating nuclease expression cassette according to any one of claims 1 to 12, wherein the expression cassette comprises more than one protein degradation signal.

14. A pharmaceutical composition comprising a self-modulating nuclease expression cassette according to any one of claims 1 to 13 and one or more of a carrier, suspending agent, and/or excipient.

15. The pharmaceutical composition according to claim 14, wherein the expression is in a non-viral delivery system.

16. The pharmaceutical composition according to claim 15, wherein the non-viral delivery system is a lipid nanoparticle.

17. A viral vector comprising self-modulating nuclease expression cassette according to any one of claims 1 to 13.

18. A recombinant AAV useful for gene editing comprising an AAV capsid and a vector genome packaged in the AAV capsid, said vector genome comprising:
(a) an expression cassette according to any of claims 1 to 13, and
(b) AAV inverted terminal repeat required for packaging the expression cassette into the capsid.

19. A composition comprising a viral vector according to claim 17 or a recombinant AAV according to claim 18 and one or more of a carrier, diluent, and/or excipient.

20. A self-inactivating meganuclease expression cassette comprising a nucleic acid sequence comprising sequence encoding a meganuclease fused to at least one protein degradation sequence and regulatory sequences which direct expression of the meganuclease and protein degradation signal as fusion protein in a host cell.

21. The self-inactivating meganuclease expression cassette according to claim 20, wherein the protein degradation signal is 10 amino acids to 50 amino acids in length.

22. The self-inactivating meganuclease expression cassette according to claim 20, wherein the protein degradation sequence is a PEST sequence of about 42 amino acids in length.

23. The self-inactivating nuclease expression cassette according to any one of claims 20 to 22, wherein the expression cassette comprises more than one protein degradation signal.

24. A pharmaceutical composition comprising a self-modulating nuclease expression cassette according to any one of claims 20 to 23 and one or more of a carrier, suspending agent, and/or excipient.

25. The pharmaceutical composition according to claim 24, wherein the expression is in a non-viral delivery system.

26. The pharmaceutical composition according to claim 25, wherein the non-viral delivery system is a lipid nanoparticle.

27. A viral vector comprising self-modulating nuclease expression cassette according to any one of claims 20 to 23.

28. A recombinant AAV useful for gene editing comprising an AAV capsid and a vector genome packaged in the AAV capsid, said vector genome comprising:
(a) an expression cassette according to any of claims 20 to 23, and
(b) AAV inverted terminal repeat required for packaging the expression cassette into the capsid.

29. A composition comprising a viral vector according to claim 26 or a recombinant AAV according to claim 28 and one or more of a carrier, diluent, and/or excipient.

30. A method for editing of a targeted gene, said method comprising delivering a self-modulating nuclease expression cassette according to any one of claims 1 to 13, a composition according to any of claims 14 to 16, a viral vector according to claim 17, or an rAAV according to claim 28.

31. A method for editing of a targeted gene, said method comprising delivering an nuclease expression cassette according to any one of claims 20 to 23, a pharmaceutical composition according to any one of claims 24 to 26, a viral vector according to claim 27 or an rAAV according to claim 28.
